SEQUENCE LISTING (28 sequences)

SEQ ID NO: 1 - DNA, 20 bp, Artificial Sequence
  MYH6 forward primer
  misc_feature (1)..(20)
attgctgaaaccgagaatgg

SEQ ID NO: 2 - DNA, 20 bp, Artificial Sequence
  MYH6 reverse primer
  misc_feature (1)..(20)
cgctccttgaggttgaaaag

SEQ ID NO: 3 - DNA, 20 bp, Artificial Sequence
  MYH7 forward primer
  misc_feature (1)..(20)
gcatcatggacctggagaat

SEQ ID NO: 4 - DNA, 20 bp, Artificial Sequence
  MYH7 reverse primer
  misc_feature (1)..(20)
atccttgcgttgagagcatt

SEQ ID NO: 5 - DNA, 18 bp, Artificial Sequence
  ACTC1 forward primer
  misc_feature (1)..(18)
gctctgggctggtgaagg

SEQ ID NO: 6 - DNA, 20 bp, Artificial Sequence
  ACTC1 reverse primer
  misc_feature (1)..(20)
ttctgacccatacccaccat

SEQ ID NO: 7 - DNA, 24 bp, Artificial Sequence
  TNNT2 forward primer
  misc_feature (1)..(24)
tgcaggagaagttcaagcagcaga

SEQ ID NO: 8 - DNA, 24 bp, Artificial Sequence
  TNNT2 reverse primer
  misc_feature (1)..(24)
agcgaggagcagatctttggtgaa

SEQ ID NO: 9 - DNA, 20 bp, Artificial Sequence
  MYL2 forward primer
  misc_feature (1)..(20)
caacgtgttctccatgttcg

SEQ ID NO: 10 - DNA, 20 bp, Artificial Sequence
  MYL2 reverse primer
  misc_feature (1)..(20)
gtcaatgaagccatccctgt

SEQ ID NO: 11 - DNA, 20 bp, Artificial Sequence
  GATA4 forward primer
  misc_feature (1)..(20)
gcggcctctacatgaagctc

SEQ ID NO: 12 - DNA, 20 bp, Artificial Sequence
  GATA4 reverse primer
  misc_feature (1)..(20)
cttccgttttctggtttgga

SEQ ID NO: 13 - DNA, 20 bp, Artificial Sequence
  GJA1 forward primer
  misc_feature (1)..(20)
caatcacttggcgtgacttc

SEQ ID NO: 14 - DNA, 20 bp, Artificial Sequence
  GJA1 reverse primer
  misc_feature (1)..(20)
cctccagcagttgagtaggc

SEQ ID NO: 15 - DNA, 23 bp, Artificial Sequence
  RYR2 forward primer
  misc_feature (1)..(23)
acagcacaagccattctgcaaga

SEQ ID NO: 16 - DNA, 24 bp, Artificial Sequence
  RYR2 reverse primer
  misc_feature (1)..(24)
atgtaatccagcccacccagacat

SEQ ID NO: 17 - DNA, 20 bp, Artificial Sequence
  SERCA2A forward primer
  misc_feature (1)..(20)
tgagacgctcaagtttgtgg

SEQ ID NO: 18 - DNA, 20 bp, Artificial Sequence
  SERCA2A reverse primer
  misc_feature (1)..(20)
tcatgcacagggttggtaga

SEQ ID NO: 19 - DNA, 20 bp, Artificial Sequence
  CACNA1C forward primer
  misc_feature (1)..(20)
tttggtccatggtcaatgag

SEQ ID NO: 20 - DNA, 19 bp, Artificial Sequence
  CACNA1C reverse primer
  misc_feature (1)..(19)
gcattggcattcatgttgg

SEQ ID NO: 21 - DNA, 20 bp, Artificial Sequence
  SCN5A forward primer
  misc_feature (1)..(20)
gggcaatgtctcagccttac

SEQ ID NO: 22 - DNA, 20 bp, Artificial Sequence
  SCN5A reverse primer
  misc_feature (1)..(20)
catcagccagcttcttcaca

SEQ ID NO: 23 - DNA, 20 bp, Artificial Sequence
  KCNH2 forward primer
  misc_feature (1)..(20)
ccttcgacctgctcatcttc

SEQ ID NO: 24 - DNA, 20 bp, Artificial Sequence
  KCNH2 reverse primer
  misc_feature (1)..(20)
tgagtagcgatccagcttcc

SEQ ID NO: 25 - DNA, 20 bp, Artificial Sequence
  KCNQ1 forward primer
  misc_feature (1)..(20)
cgcctgaaccgagtagaaga

SEQ ID NO: 26 - DNA, 20 bp, Artificial Sequence
  KCNQ1 reverse primer
  misc_feature (1)..(20)
aaggagagcagctggtgaag

SEQ ID NO: 27 - DNA, 19 bp, Artificial Sequence
  GAPDH
  misc_feature (1)..(19)
agggatctcgctcctggaa

SEQ ID NO: 28 - DNA, 19 bp, Artificial Sequence
  GAPDH reverse primer
  misc_feature (1)..(19)
agggatctcgctcctggaa

(19) United States
(12) Patent Application Publication
(10) Pub. No.: US 2019/0365951 A1
(43) Pub. Date: Dec. 5, 2019

(54) HUMAN CARDIAC TISSUE CONSTRUCT, RELATED METHODS AND USES

(71) Applicants: FUNDACIÓ INSTITUTE DE BIOENGINYERIA DE CATALUNYA (IBEC), Barcelona (ES); FUNDACIÓ CENTRE DE MEDICINA REGENERATIVA DE BARCELONA (CMRB), L'Hospitalet de Llobregat (ES); FUNDACIÓ PRIVADA INSTITUCIÓ CATALANA DE RECERCA I ESTUDIS AVANÇATS (ICREA), Barcelona (ES); UNIVERSITAT POLITÈCNICA DE CATALUNYA, Barcelona (ES)

(72) Inventors: Elena Martínez Fraiz, Barcelona (ES); Ángel Raya Chamorro, L'Hospitalet de Llobregat (ES); Raimon Jané Campos, Barcelona (ES); Maria Valls Margarit, Barcelono (ES); Olalla Iglesias García, L'Hospitalet de Llobregat (ES)

(21) Appl. No.: 16/431,593

(22) Filed: Jun. 4, 2019

(30) Foreign Application Priority Data
Jun. 4, 2018 (EP) 18 382 391.3

(51) Int. Cl. A61L 27/38; A61L 27/24; A61L 27/56; C12N 5/077; G01N 33/50

(52) U.S. Cl. CPC A61L 27/3873; C12N 2529/00; A61L 27/3834; A61L 27/3826; A61L 27/24; A61L 27/3886; A61L 27/56; C12N 5/0657; G01N 33/5061; G01N 33/5014; A61L 2430/20; C12N 2501/40; C12N 2506/45; C12N 2533/54; A61L 27/3895

(57) ABSTRACT

The present disclosure relates to a human cardiac tissue construct, to the method for producing thereof and its uses in disease modelling, compound screening and properties evaluation, and/or therapeutic uses in heart regeneration. It further relates to a perfusion bioreactor with electrical stimulation capabilities and its use in the production of said human cardiac tissue construct. In still a further aspect, the disclosure provides a method for the non-destructive evaluation of electrophysiological activity in a cellular construct, such as a cardiac tissue construct of the disclosure.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 370085_401_SEQUENCE_LISTING. The text file is 7 KB, was created on Jun. 3, 2019, and is being submitted electronically via EFS-Web.

### BACKGROUND

### Technical Field

The present disclosure relates to the field of cardiac tissue engineering. Specifically it relates to a human cardiac tissue construct, to the method for producing thereof and its uses in disease modelling, compound screening and properties evaluation, and/or therapeutic uses in heart regeneration. It further relates to a perfusion bioreactor with electrical stimulation capabilities and its use in the production of said human cardiac tissue construct. In still a further aspect, the disclosure provides a method for the non-destructive evaluation of electrophysiological activity in a cellular construct, such as a cardiac tissue construct of the disclosure.

### Description of the Related Art

Cardiac tissue engineering aims at producing constructs with structural, physiological and functional properties resembling human native cardiac tissue. Ultimately, such in vitro models will find applications in disease modeling, drug screening and toxicology, and replacing or regenerating damaged heart tissue. The structural organization of the cardiac tissue is complex, comprising a vast array of diverse cell types (including fibroblasts, cardiac myocytes, and smooth muscle, pacemaker, and endothelial cells), arranged in a precise and stereotypical architecture to ensure that the critical function of pumping blood throughout the body is maintained (Chien, K. R. et al, 2008). Efficient blood pumping requires the ˜5 billion cardiomyocytes that make up an average human adult heart contracting and relaxing in a coordinated and timely order. Multi-scale structural features characteristic of the heart's intracellular and intercellular organization enable the necessary coordination for the entire heart muscle to form a functional syncytium (Hunter, P. J. et al, 2003). This results in electrochemical processes of such magnitude that generate voltage potentials of ˜1 mV, easily recorded on the body surface. That the shape of these voltage potential waves (electrocardiogram, ECG) be a reliable indicator of cardiac performance, used on a routine basis in clinical cardiology for over a century (Fermini, B. & Fossa, et al, 2003), clearly attests to the intimate dependence of structure and function in the heart.

Due to the high dependence found between cardiac muscle structural organization and its function, it has been hypothesized that growing cardiac constructs in engineering systems mimicking relevant physicochemical stimuli found in vivo would be advantageous to achieve tissue-like properties (Fleischer, S. et al, 2017). In the last years, tissue engineering methods have significantly advanced in generating functional 3D cardiac constructs (Zimmermann, W. H. et al, 2002; Shimizu, T. et al, 2002; Radisic, M. et al, 2004). A key issue that has deserved much attention in this field is the degree of cardiomyocyte maturation achieved within the engineered cardiac constructs (recently reviewed in (Parsa, H. et al, 2016)). This issue is particularly important when using cardiomyocytes derived from pluripotent stem cells (PSC), which are typically immature, fetal-like under standard 2D differentiation conditions (Feric, N. T. & Radisic, M., 2016). Human engineered cardiac constructs developed thus far recapitulate some of the structural complexity and electromechanical functionality of the native myocardium, leading to improved performance compared to standard 2D in vitro cultures (Schaaf, S. et al, 2011; Nunes, S. S. et al, 2013; Thavandiran, N. et al, 2013; Ma, Z. et al, 2015). Exogenous stimuli such as mechanical and electrical signals have been shown to further improve the electrophysiological properties, the cellular and ultrastructural organization, and the expression of cardiac specific proteins of cardiac constructs (Radisic, M. et al, 2004; Tandon, N. et al, 2009; Godier-Furnemont, A. F. et al, 2015). These strategies have resulted in microengineered models of human cardiac muscle, which emerged as promising platforms for preclinical toxicology and drug screening assays (Hansen, A. et al, 2010; Mathur, A. et al, 2015; Amano, Y. et al, 2016). However, microtissues are minimal units with some cardiac functionality, but they are inherently limited in size and cannot fully capture the complexity of the native cardiac tissue structure (Kurokawa, Y. K. & George, S. C., 2016). Unfortunately, the production of human macroscale tissues displaying in vivo-like complexity and, therefore, tissue-like functionality, is still an unmet challenge (Fleischer, S. et al, 2017).

The design of tissue engineering constructs in the macroscale (greater than 300 μm in thickness) is met with the challenge that effective mass transfer cannot rely on passive diffusion alone (Lovett, M. et al, 2009). This is critical for cardiac tissue constructs due to the comparatively high metabolic demand of cardiac muscle cells, which requires a controlled microenvironment with the appropriate supply of oxygen and nutrients (Carrier, R. L. et al, 2009; Radisic, M. et al, 2004; Radisic, M. et al, 2008). Perfusion bioreactor systems pioneered in Dr. Vunjak-Novakovic's laboratory have proved to be valuable in the generation of thick cardiac tissue constructs full of viable cells with aerobic metabolism (Radisic, M. et al, 2004).

Electrical stimulation, alone or in combination with mechanical loading, has been widely applied in tissue engineering to generate cardiac constructs that recapitulate some aspects of the native physiology (Nunes, S. S. et al, 2013; Godier-Furnemont, A. F. et al, 2009). Electrical pacing has also been applied to the generation of human cardiac microtissues (Nunes, S. S. et al, 2013; Thavandiran, N. et al, 2013; Xiao, Y. et al, 2016; Ruan, J. L. et al, 2016). Such microtissues are usually fabricated by using cell-laden natural-based hydrogels casted on posts (Schaaf, S. et al, 2011; Thavandiran, N. et al, 2013; Tiburcy, M. et al, 2017; Ruan, J. L. et al, 2016; Soong, P. L. et al, 2012; Fennema, E. et al, 2013; Kensah, G. et al, 2013; Zhang, D. et al, 2014; Hinson, J. T. et al, 2015; Huebsch, N. et al, 2012), or around a wire template (Nunes, S. S. et al, 2013; Xiao, Y. et al, 2014), but the delivery of nutrients and oxygen is limited by diffusion to ˜300 μm in thickness (Lovett, M. et al, 2009). To overcome such size limitation, the production of thicker cardiac constructs can be then performed by medium perfusion bioreactors, which are able to maintain cell viability over time (Radisic, M. et al, 2008). Perfusion bioreactors incorporating electrical stimulation have been previously applied for in vitro culture of murine cardiomyocyte 3D tissue structures (Barash, Y. et al, 2010; Maidhof, R. et al, 2012; Kensah, G. et al, 2011). However, this research has been rarely transferred to the human cardiac tissue engineering models (Tiburcy, M. et al, 2017; Ma, Z. et al, 2014).

Despite recent advances, in vitro generation of human heart tissue is still limited by the existing tissue engineering technologies, and thus there exists a need to obtain a macroscale cardiac construct which recapitulates the complex structure and function of the human myocardium.

### BRIEF SUMMARY

The inventors have developed an innovative method for the in vitro production of contractile human cardiac macrotissues with tissue-like functionality. They further designed and built a parallelized bioreactor able to simultaneously provide fluid perfusion and electrical stimulation to several cardiac constructs. The method for a human macroscale cardiac construct production is scalable in size, thus compatible with the fabrication of thick macrotissues. As a unique feature, this system enabled on-line monitoring of tissue function over time, providing for the first time a technology suitable for the evaluation of the electrophysiological properties of thick cardiac macrotissues. In vitro culture of hPSC-derived cardiomyocytes together with fibroblasts under electrostimulation resulted in engineered cardiac macrotissues, referred as CardioSlice constructs, displaying cardiac tissue-like properties, both at structural and functional levels.

As shown in the Examples, cardiomyocytes derived from human pluripotent stem cells (hPSC) differentiated under standard 2D conditions were seeded, together with human fibroblasts, into 3D collagen-based porous scaffolds of 10 mm in diameter and 1 to 2 mm thick. Constructs were cultured for up to 14 days in a parallelized perfusion bioreactor equipped with custom-made culture chambers endowed with electrostimulation capabilities. The constructs obtained developed into macroscopically contractile structures in which cardiomyocytes showed signs of increased cell maturation compared to those cultured under 2D conditions, and similar to those of microtissues. More importantly, continuous electrical stimulation of the cardiac macrotissues for 2 weeks promoted cardiomyocyte alignment and synchronization, and the emergence, for the first time to our knowledge, of cardiac tissue-like properties. These translated into spontaneous electrical activity that could be readily measured on the surface of the obtained constructs as ECG-like signals, and a response to proarrhythmic drugs that was predictive of their effect in human patients.

The first aspect of the disclosure relates to a method for producing a human tridimensional macroscale cardiac construct, wherein said method comprises the following steps:

(i) differentiating human pluripotent stem cells or adult cardiac stem cells into contracting cardiomyocytes,

(ii) suspending the cardiomyocytes together with human fibroblasts to obtain a mixed cell suspension;

(iii) seeding the mixed cell suspension into a collagen-based porous scaffold,

(iv) optionally, culturing the seeded scaffold under conditions that allow cell attachment to the scaffold, and

(v) transferring the cardiac construct to a bioreactor and culturing it under perfusion with electrical stimulation for cardiomyocyte maturation,

thereby obtaining a human tridimensional macroscale cardiac construct displaying spontaneous beating;

wherein a macroscale construct has a thickness greater than 300 μm.

Another aspect of the present disclosure refers to a human tridimensional macroscale cardiac construct obtained or obtainable by a method as described herein.

In a further aspect, the disclosure refers to a cardiac construct as defined herein, for use in the treatment of a human subject having cardiac damage. In a related aspect, the disclosure refers to a method of treating a subject having cardiac damage by administration of a therapeutically effective amount of the cardiac cell construct described herein.

In an additional aspect, the present disclosure refers to the in vitro use of a cardiac construct as described herein, for the screening or evaluation of compounds, such as drugs, on cardioprotective or cardiotoxic properties.

In another aspect, the present disclosure refers to the in vitro use of a cardiac construct as described herein, for cardiac disease modeling.

Moreover, in a further aspect, the disclosure provides a bioreactor, preferably a parallelized bioreactor, equipped with electrodes for electrically stimulating cells (e.g., hPSC or cardiac stem cells-derived cardiomyocytes in a cell construct as described herein) during perfusion in culture. Preferably, said bioreactor further contains means for measuring and/or recording electrical signals.

In still a further aspect, the disclosure provides a method for the non-destructive evaluation of electrophysiological activity in a cardiac construct, said method comprising the use of a bioreactor as described herein enabling the measuring and/or recording of electric signals, such as ECG-like signals.

### BRIEF DESCRIPTION OF THE SEVERAL VIEWS OF THE DRAWINGS

FIG. 1. Parallelized perfusion bioreactor for the generation of CardioSlice constructs. (a) 3D modelling of the electric field generated in our custom-made perfusion chamber when applying a differential voltage of 5 V. Electrode configuration (red and blue cylinders in the geometry, positive and negative, respectively) and predicted electric field and current density values are displayed both in 3D and top views. Black arrowheads indicate the direction of the electric field. Plots show electric field and current density values at the positions where cells are seeded in the scaffold (central circle in the top view) (from x=−2.5 to x=2.5). (b) Images of the perfusion chamber with electrical stimulation fabricated. The cardiac construct is represented in the cross-section (pink), which is held in place by two gaskets (silicone: blue; polypropylene: yellow). Black rectangles represent graphite electrodes; bright yellow dot represents the gold electrode. (c) Voltage measurements in the perfusion chamber with electrical stimulation. Measured electric field values coincide with the values predicted by the model. (d) Overall view of the parallel bioreactor system: medium reservoir (1), luer manifold (2), de-bubblers (3), flow restrictors (4), perfusion chambers (5), gas exchanger (6), and peristaltic pump (7). The bioreactor supports the culture of up to four cardiac constructs simultaneously. Two different perfusion chambers are used to either electrically stimulate cardiac constructs while culturing or not.

FIG. 2. Morphology and ultrastructural organization of human cardiac macrotissues. (a) Representative cross-sections of cardiac constructs after 14 days of culture without (Control) or with electrical stimulation (CardioSlice). hiPSC-derived cardiomyocytes positive for cardiac troponin (cTnT; green) and Phalloidin (Ph; red) were detected in the scaffold. Nuclei were stained with 4′, 6-diamidino-2-phenylindole (DAPI; blue). Cardiac macrotissues were analyzed by Second Harmonic Generation for the detection of collagen (Col; violet). Scale bars: 500 μm, higher magnifications: 100 μm, 50 μm and 10 μm, respectively. (b) Standard 2D culture of hiPSC-derived cardiomyocytes in monolayer. Cardiomyocytes were positive for cardiac troponin (cTnT; green) and Phalloidin (Ph; red). Scale bar: 50 μm. (c) Ultrastructural analysis of hiPSC-derived cardiac macrotissues. Representative transmission electron microscopy images of cardiac macrotissues were shown after 7 and 14 days of culture. Scale bars: 1 μm (bundles) and 0.5 μm (sarcomere width and cell junctions). (d) Morphometric analysis showing sarcomere width (***p<0.001, n=2 per group, Mann Whitney U test). Data are expressed as mean±standard deviation (SD).

FIG. 3. Functional assessment of human cardiac macrotissues. (a) Top view images of the human cardiac macrotissues after 7 and 14 days of culture, both without (Control) or with (CardioSlice) electrical stimulation. (b) Contraction amplitude analysis of Control and CardioSlice constructs after 7 and 14 days of culture. Area oscillation is represented over time. (c) Bar chart showing the percentage of Fractional Area Change (FAC) for each cardiac macrotissue (average±SEM) (***p<0.001; n≥3 per group, Mann Whitney U test). (d) Maximum Capture Rate (MCR) of Control and CardioSlice constructs after 7 and 14 days of culture (average±SD) (***p<0.001, n≥3 per group, Mann Whitney U test). (e) Representative velocity maps of beating human cardiac macrotissues after 14 days of culture. Red colors and longer arrows represent higher velocities, while blue colors and shorter arrows represent lower velocities. Scale bars=2.5 mm. (f) Analysis of the alignment between the direction of the electric field and the beating direction of human cardiac macrotissues beating. The order parameter <cos 2θ> was used, with values close to 0 meaning random distribution and values close to 1 meaning parallel alignment (average±SD) (***p<0.001; n=2 per group, Mann Whitney U test). (g) Strain rate of representative macrotissues over time. Positive values correspond to contractions, whereas negative values correspond to relaxations. (h) Strain rate per beat (average±SD) (**p<0.01; n>4 per group, one-way ANOVA).

FIG. 4. Electrocardiogram (ECG)-like signals generated by human cardiac macrotissues during spontaneous beating. (a) Representative bioelectrical signals of non-stimulated (Control) and electrically stimulated (CardioSlice) human cardiac macrotissues during spontaneous beating (left panel) and patterns estimated by signal averaging (right panel). In this example, bioelectric signals were bandpass filtered (zero-phase fourth-order Butterworth filter with cut-off frequency of 0.2 and 40 Hz, respectively). (b, c, d) Dot charts showing the QRS complex duration (b), the QT interval duration (c) and the amplitude of the QRS complex (d) from the signals generated by human cardiac macrotissues, relative to electrical pacing conditioning (average±SD) (***p<0.001, n=3 per group, F-test). (e, f) Representative traces of ECG-like signals recorded for CardioSlice constructs at baseline (first minute), and after isoproterenol (e) and carbachol (f) treatments (10 min incubation). Bar charts show the effect of isoproterenol and carbachol on the beat rate, relative to baseline (average±SD) (***p<0.001, Mann-Whitney U test).

FIG. 5. Effects of sotalol on the electrophysiology of CardioSlice constructs at day 14 of culture. (a) Instantaneous beating rate and representative traces of ECG28 like signals for Control constructs upon 10 min treatment with sotalol. I and II (brown shading) indicate the time lapse from which the traces have been obtained. (b) Instantaneous beating rate and representative traces of ECG-like signals for CardioSlice constructs upon 10 min treatment with sotalol. I and II (brown shading) indicate the time lapse from which the traces have been obtained. (c) QT interval prolongation in CardioSlice constructs, estimated by signal averaging at basal conditions and after 5 and 10 min of incubation with sotalol. (d) Instantaneous beating rate and representative traces of ECG-like signals for CardioSlice constructs upon 30 min treatment with sotalol. I, II and III (brown shading) indicate the time lapse from which the traces have been obtained.

FIG. 6. Matriderm® collagen-elastin scaffold characterization. (a-c) Selective imaging of Matriderm® scaffold by second harmonic generation (SHG) and two-photon excited fluorescence (TPEF, autofluorescene). (a) Matriderm® imaged by SHG. (b) Matriderm® imaged by two-photon excited fluorescence (TPEF). (c) Overlay image of A and B. (d) Scanning electron micrograph of Matriderm®. (e) Porosity analysis: Feret's diameter has been calculated for scaffold pores, and values have been fitted in a single peak (17 μm). (f) Matriderm® stiffness in compression at room temperature (RT) and at 37° C. (average±SD; n=3 per group). Young's Modulus (E) has been determined from the slope of stress-strain curves (*p<0.05; Student's t-test). Scale bars: 100 μm.

FIG. 7. Cardiac differentiation potential of hiPSC. (a) Schematics of the protocol for the differentiation of cardiomyocytes from hiPSC with modulators of Wnt signalling pathway. Bright field images of the cell morphology at day −3, day −2, day 0, day 5 and day 20 of differentiation are shown. (b) Flow cytometry analysis of cardiomyocytes differentiated from hiPSC at day 19 of differentiation. Cells were analyzed for cardiac troponin I (cTnI) and myosin heavy chain (MHC) expression. High purity of cardiomyocytes of over 70% was obtained (n=3). (c) Immunofluorescence detection of cardiac proteins. Cardiomyocytes were selected at day 19 of differentiation and stained for cTnI (Cy2: green), α-actinin sarcomeric (AAS) (Cy3: red) and connexin-43 (Cx43) (Cy2: green). Nuclear staining was performed with DAPI. (d) Expression of cardiac markers in differentiated hiPSC. Cardiac gene expression was determined by quantitative PCR at day 0, day 20 and day 35 of differentiation. Up-regulation of cardiac specific genes and channel markers was detected in differentiated cells (n=2), although expression values still differed from adult heart (AH) tissue. Data are expressed as mean±SD. Scale bars: 200 μm (a), 25 μm (c), 2 μm (e); higher magnification: 1 (e).

FIG. 8. Scheme of the strategy to generate CardioSlice constructs. Cardiomyocytes derived from human induced pluripotent stem cells (hiPSC-CMs) were selected at day 20 of differentiation and mixed with human foreskin fibroblasts (HFF). Then, the cell suspension was seeded inside the scaffold by one-way perfusion at 1 ml/min. Cardiac constructs were installed in the bioreactor, and they were cultured either with (CardioSlice constructs) or without electrical stimulation (Control constructs) for 14 days. Finally, ECG-like signals generated by cardiac constructs were recorded in real time.

FIG. 9. Generation of functional and structurally organized rat cardiac macrotissues after 7 days of culture in a perfusion bioreactor. (a) Immunostaining of engineered rat cardiac macrotissues. Representative cross-sections and higher magnification images, where cardiomyocytes' distribution along the scaffold and their sarcomeric organization and alignment is shown. DAPI: 4′,6-diamidino-2-phenylindole; cTNI: cardiac troponin I; ASA: α-sarcomeric actin. Scale bars: 400 μm, higher magnifications: 100 and 50 μm. (b) Representative images of cardiomyocytes' ultrastructural organization (sarcomeric structure and cellular junctions) when cultured with or without electrical stimulation. Scale bar: 0.5 (c) Morphometric analysis showing sarcomere width (measured as indicated by square brackets in (b)). ES: Electrical stimulation; NRV: neonatal rat ventricle (average±SD) (***p<0.001; n≥2 per group, Mann Whitney U test). (d) Contraction amplitude analysis of control and electrostimulated rat cardiac macrotissues by means of Fractional Area Change (FAC). The area in pixels of each construct was obtained through custom MATLAB program (black dashed line), and its oscillation was represented over time (relative to the highest number of pixels recorded). Bar chart shows the fold induction relative to controls mean (n=3 per group). Scale bar: 0.25 μm. ES: Electrical stimulation. (e) Excitation threshold (ET) of control and electrostimulated rat cardiac macrotissues (average±SD) (n=3 per group). (f) Maximum Capture Rate (MCR) of control and electrostimulated rat cardiac macrotissues (average±SD) (n=3 per group). ES: Electrical stimulation.

FIG. 10. Endogenous gene expression of cardiac specific markers measured by qRT-PCR. Expression of cardiac markers in: 34-day-old cardiomyocyte monolayer standard culture (2D); non-stimulated cardiac constructs (Control); electrically stimulated cardiac constructs (CardioSlice); fetal heart (FH) and adult heart (AH) (n≥3 per group). MYH7: β-myosin heavy chain; ACTC1: cardiac muscle alpha actin; TNNT2: cardiac troponin T; MYL2: cardiac ventricular myosin light chain 2; GATA4: gata binding protein 4; GJIA: gap junction protein alpha 1; RYR2: ryanodine receptor 2; SERCA2A: Sarcoplasmic Reticulum Ca2+-ATPase isoform 2a; CACNA1C: voltage-gated L type calcium channel alpha 1C subunit; SCN5A: sodium voltage-gated channel alpha subunit 5; KCNH2: potassium voltage-gated channel subfamily h member 2; KCNQ1: potassium voltage-gated channel subfamily Q member 1. Data are expressed as mean±standard error of the mean (SEM).

FIG. 11. Chronotropic responses of human cardiac macrotissues to the proarrhythmic drug sotalol. Changes in the beat rate were analyzed through custom MATLAB program at different incubation times (from 0 min to 40 min). The area in pixels of each construct was obtained, and its oscillation was represented over time (relative to the highest number of pixels recorded). (a) Sotalol effects on non-stimulated cardiac constructs (Control). (b) Sotalol effects on electrically stimulated cardiac constructs (CardioSlice). Initially, a negative chronotropic effect can be observed, followed by a progressive emergence of cardiac arrhythmias. * Increased relaxation time; # Increased beating rate.

### DETAILED DESCRIPTION

In a first aspect, the disclosure refers to a method for producing a human tridimensional macroscale cardiac construct, wherein said method comprises the following steps:

(i) differentiating human pluripotent stem cells or adult cardiac stem cells into contracting cardiomyocytes,

(ii) suspending the cardiomyocytes together with human fibroblasts to obtain a mixed cell suspension;

(iii) seeding the mixed cell suspension into a collagen-based porous scaffold,

(iv) optionally, culturing the seeded scaffold under conditions that allow cell attachment to the scaffold, and

(v) transferring the cardiac construct to a bioreactor and culturing it under perfusion with electrical stimulation for cardiomyocyte maturation,

thereby obtaining a human tridimensional macroscale cardiac construct displaying spontaneous beating;

wherein a macroscale construct has a thickness greater than 300 μm.

Human pluripotent stem cells (hPSCs) may be used in step (i). These are stem cells having pluripotency which enables the cells to differentiate into derivatives of the three main embryo germ layers (endoderm, ectoderm, and mesoderm), and also possess self-renewing ability, which enables them to proliferate indefinitely in vitro. Examples of the pluripotent stem cells include, but are not limited to human embryonic stem (hES) cells, preferably obtained from existing hES cell lines generated without destroying a human embryo (e.g., Chung et al., 2008), or from parthenogenetic activation of an oocyte in the absence of sperm (WO 2003/046141), and induced pluripotent stem (iPSCs) cells. Alternatively, adult cardiac stem cells may also be used in step (i) for differentiation into cardiomyocytes. A number of different cardiac stem cells and stem cell lines have been described in the art, including those described in WO 99/49015, WO 2005/012510, WO 2006/052925, WO 02/09650, WO 02/13760, WO 03/103611, WO 2007/100530, WO 2009/073616, WO 2011/057249, WO 2011/057251, WO 2012/048010, WO 2006/093276, WO 2009/136283 and WO 2014/141220.

Preferably, the hPSCs in step (i) are human iPSCs. iPSCs are pluripotent cells obtained by reprogramming adult somatic cells by transient overexpression of specific nuclear factors. Takahashi et al. (Takahashi et al. 2007) disclosed for the first time methods for reprogramming differentiated cells and establishing an induced pluripotent stem cell having similar pluripotency and growing abilities to those of an ES cell. Takahashi et al. described various different nuclear reprogramming factors for differentiated fibroblasts, which include products of the following four gene families: an Oct family gene; a Sox family gene; a KIf family gene; and a Myc family gene.

The iPSCs which may be used in the method of the disclosure can be obtained for instance by the methods described by Takahashi et al. (Takahashi et al. 2007). Alternatively, other methods could be used, such as those using non-integrative Sendai virus (Ban H et al., 2011), episomal plasmids (Yu J. et al., 2009), or mRNA transfection (Warren L. et al., 2010). Moreover, the reprogrammed adult somatic cells may be from different cell types and tissue origins, including but not limited to dermal fibroblasts, epidermal keratinocytes, peripheral blood mononuclear cells, urine sediment cells, and mesenchymal stromal cells. Preferably, said human iPSCs are derived from foreskin dermal fibroblasts. More preferably, said iPSCs are from the human FIPS Ctr11-mR5F-6 cell line (National Stem Cell Bank, Institute of Health Carlos III, Spanish Ministry) used in the Examples.

Various protocols for the obtaining of cardiomyocytes from hPSCs have been described and are well known in the art. Differentiation into cardiomyocytes from hESCs may for instance be conducted via embryoid bodies (EBs) in a medium containing fetal calf serum. The differentiation protocol may optionally comprise the addition of growth factors, including but not limited to fibroblast growth factor 2 (FGF2), transforming growth factor beta (TGFbeta), vascular endothelial growth factor (VEGF); and the addition of Gsk3 inhibitors and/or Wnt inhibitors (Graichen R. et al, 2008; Yang L. et al, 2008; Kattman S J. et al, 2011; Mohr J C. et al, 2010; Azarin S M. et al, 2012; Lian X. et al, 2012; Zhang J. et al, 2012).

Preferably, hPSCs differentiation into cardiomyocytes is conducted in monolayer culture. For instance, said differentiation protocol may be based on TGFβ superfamily growth factors, such as protocol 1 (GiAB) in Lian X. et al., 2013, which relies upon treatment of undifferentiated hPSCs with Gsk3 inhibitor in mTeSR1, followed by Activin A and BMP4 in RPMI/B27-insulin. It also can be based on employing small molecule activators of canonical Wnt signalling followed by shRNA of β-catenin expression (protocol 2, GiSB) or small molecule inhibitors of Wnt signaling (protocol 3, GiWi) in a growth factor-free system (Lian X. et al., 2013). The small molecule methods (protocols 2 and 3) use the sequential treatment of Gsk3 inhibitors and Wnt signaling inhibitors (or inducible expression of β-catenin shRNA) to stimulate cardiogenesis.

In a preferred embodiment, cardiomyocyte differentiation in step (i) is conducted in monolayer culture with a method comprising the addition of a Gsk3 inhibitor (e.g., CHIR99021, Stemgent) followed by the addition of a Wnt signalling inhibitor (e.g., IWP4, Stemgent) generally at day 3 of differentiation. Preferably, the differentiation protocol is as described in the Examples, where spontaneously contracting cardiomyocytes are obtained at around day 8. These cardiomyocytes contract or beat spontaneously and this is visible at a macroscopic level.

Contracting cardiomyocytes are preferably obtained from beating clusters in the monolayer which are disaggregated, typically by trypsin-EDTA digestion, prior to suspension in step ii). In a preferred embodiment, optionally in combination with one or more of the features or embodiments described herein, the contracting cardiomyocytes used in step ii) are obtained from beating clusters around day 20, preferably at day 20 or onwards, of differentiation with the protocol as described in the Examples.

The differentiated cardiomyocytes, or a substantially pure population of cardiomyocytes, obtained in step (i) may further be characterized by expressing the markers cardiac Troponin T (cTnT) and/or myosin heavy chain (MHC).

The marker profile of the cardiomyocytes, or the substantially pure cardiomyocytes population, can be further defined by the presence and/or absence of additional markers, or by a specific profile of a combination of present and absent markers. In each case, the specific combination of markers may be present as a particular profile within a population of cells and/or a particular profile of markers on individual cells within the population.

In one particular embodiment, the cardiomyocytes and/or the substantially pure population of cardiomyocytes express one or more of cTnT and MHC at a detectable level. In a further embodiment, the cardiomyocytes and/or the substantially pure population of cardiomyocytes express both cTnT and MHC at a detectable level.

In one particular embodiment, at least about 80%, 85%, 90%, 95%, 97% or 100% of the cardiomyocytes in the substantially pure cardiomyocytes population express cTnT and/or MHC at a detectable level. In another particular embodiment, at least about 70%, 75%, 80%, 85%, 90%. 95% or 100% of the cardiomyocytes in the substantially pure cardiomyocytes population express cTnT and MHC at a detectable level. In any of these embodiments, the indicated expression levels are for instance when expression is determined by flow cytometry or fluorescence-activated cell sorting (FACS) analysis.

The cardiomyocytes and/or cells of the substantially pure population of cardiomyocytes may also express one or more, i.e., 1, 2, 3, 4, 5, 6, 7, 8, 9, 10 or all of the markers selected from the group consisting of homeobox protein NKX2.5, GATA-4, GATA-6, MESP-1, ANF, SIRPA, myosin light chain 2-atrial, myosin light chain 2-ventricular, β-myosin heavy chain, sarcomeric α-actinin, and titin.

The term “marker” as used herein encompasses any biological molecule whose presence, concentration, activity, or phosphorylation state may be detected and used to identify the phenotype of a cell.

The term “expressed” is used to describe the presence of a marker within a cell. In order to be considered as being expressed, a marker must be present at a detectable level. By “detectable level” is meant that the marker can be detected using one of the standard laboratory methodologies such as PCR, blotting, immunofluorescence, ELISA, flow cytometry or FACS analysis. Preferably, determination of protein expression levels at the cell surface is conducted by flow cytometry or FACS. “Expressed” may refer to, but is not limited to, the detectable presence of a protein, phosphorylation state of a protein or an mRNA encoding a protein. A gene is considered to be expressed by a cell or cell population if expression can be reasonably detected after 30 PCR cycles, preferably after 37 PCR cycles, which corresponds to an expression level in the cell of at least about 100 copies per cell. The terms “express” and “expression” have corresponding meanings. At an expression level below this threshold, a marker is considered not to be expressed. The comparison between the expression level of a marker in a cardiomyocyte, and the expression level of the same marker in another cell, for example on pluripotent stem cells or fibroblasts which can be used as control cells, may be conducted by comparing the two cell types that have been isolated from the same species.

In an alternative embodiment, the cardiomyocytes and/or the substantially pure population of cardiomyocytes are considered to express a marker if the expression level of the marker is greater in the cells of the disclosure than in a control cell, for example in hPSCs or human fibroblasts. By “greater than” in this context, it is meant that the level of the marker expression in the cell population of the disclosure is at least 2-, 3-, 4-, 5-, 10-, 15-, 20-fold higher than the level in the control cell.

In step (ii), the method of the disclosure comprises suspending the cardiomyocytes together with human fibroblasts, preferably in culture medium, to obtain a mixed cell suspension.

By “cell growth medium” or “cell culture medium” it is meant a nutritive solution for culturing or growing cells. The ingredients that compose such media may vary depending on the type of cell to be cultured. In addition to nutrient composition, osmolarity and pH are considered important parameters of culture media.

The cell growth medium comprises a number of ingredients well known by the man skilled in the art, which typically for the culturing of eukaryotic cells includes amino acids, vitamins, organic and inorganic salts, sources of carbohydrate, lipids, trace elements (CuSO4, FeSO4, Fe(NO3)3, ZnSO4, etc.), each ingredient being present in an amount which supports the cultivation of a cell in vitro (i.e., survival and growth of cells). Ingredients may also include different auxiliary substances, such as buffer substances (like sodium bicarbonate, Hepes, Tris, etc.), oxidation stabilizers, stabilizers to counteract mechanical stress, protease inhibitors, animal growth factors, plant hydrolyzates, anti-clumping agents, anti-foaming agents. If required, a non-ionic surfactant, such as polypropylene glycol can be added to the cell growth medium as an anti-foaming agent. These agents are generally used to protect cells from the negative effects of aeration since, without an addition of a surfactant, the ascending and bursting air bubbles can lead to damage of those cells that are located on the surface of these air bubbles (“sparging”).

The cell growth medium is preferably an animal “serum-free medium” (SFM), which meant that the cell growth medium is ready to use, that is to say that it does not required serum addition allowing cells survival and cell growth. The cell growth medium is preferably chemically defined, but it may also contained hydrolyzates of various origins, from plant for instance. Preferably, said cell growth medium is “non-animal origin” qualified, that is to say that it does not contain components of animal or human origin (FAO status: “free of animal origin”). Several media are commercial available and can be used. Media for the culturing of eukaryotic cells include, for example: Ham's F12 Medium (Sigma, St. Louis, Mo.), Dulbecco's Modified Eagles Medium (DMEM, Sigma), RPMI (Invitrogen) or VP SFM (lnVitrogen). Preferably said culture medium is RPMI (Invitrogen) supplemented with B27 (Life Technologies) medium.

The introduction of the appropriate amount and type of fibroblasts has been reported to promote tissue organization and improve cell connectivity (Amano, Y. et al, 2016). Said fibroblasts are preferably dermal skin fibroblasts. The skin origin is not particularly limited, but foreskin fibroblasts are preferred. In said cell suspension cardiomyocytes and fibroblasts may be found at a ratio from 10:1 to 5:1, preferably at a 7:1 ratio.

In step (iii) the cell mixture is seeded in a collagen-based porous scaffold. The term “collagen-based” as used herein means that collagen is one of the main components of the scaffold. Preferably, the collagen-based porous scaffold in step (iii) is a collagen and elastin-based porous scaffold, which may be obtained for instance from bovine dermis. One advantage associated to a collagen-based scaffold is that it is a biocompatible material which will be fully degraded in the clinical setting, e.g., further to in vivo transplantation.

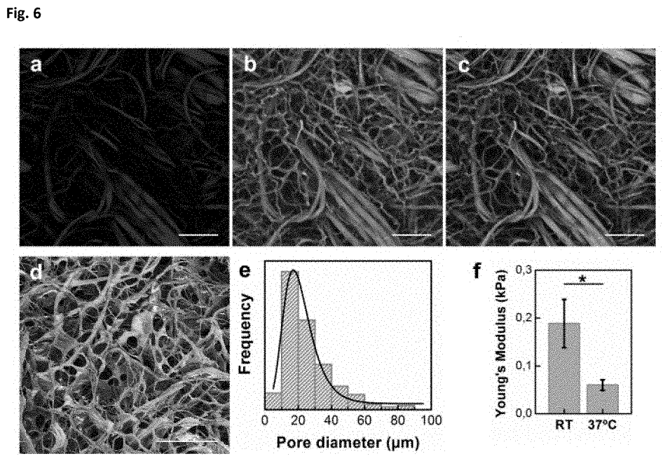

The collagen-based porous scaffold in step (iii) preferably has macropores with a mean pore size in the range of 50 to 90 μm and micropores with a mean pore size in the range of 5 to 50 μm, when the mean pore size is analyzed in dry conditions by scanning electron microscopy (SEM), e.g., under 1 mbar water pressure and without any conductive coating. More preferably, most of the pores are micropores with a mean pore size in the range between 10 and 40 μm. Most preferably, the pore size distribution is as shown in FIG. 6e.

The collagen-based porous scaffold in step (iii) is preferably a hydrated scaffold, for instance this may have been hydrated in PBS for 24h prior to cell seeding. With respect to its size, the collagen-based porous scaffold may have between 5 and 50 mm, preferably about 10 mm or about 20 mm in diameter; and between 0.5 and 3 mm, preferably between 1 and 2 mm, in thickness in the hydrated form. In a preferred embodiment, optionally in combination with one or more of the features or embodiments described herein, the collagen-based porous scaffold in step (iii) has about 10 mm of diameter and thickness of about 1 mm in the hydrated form.

In a preferred embodiment, said collagen-based porous scaffold is the collagen and elastin scaffold named Matriderm® (Medskin solutions Dr. Suwelack A G) described in Halim A S et al., 2010) which structural features are also shown in FIG. 6.

Cell seeding in step (iii) is preferably conducted by perfusion so that the cell suspension is forced to pass through the scaffold, this may be conducted for instance using a perfusion loop as described in the examples. More preferably, perfusion seeding is carried out at a flow rate of 1 ml/min. In preferred embodiments, a total amount of 5 million or more of total cells are seeded in step (iii).

In step (iv) the seeded scaffold is preferably cultured in ultralow attachment dishes (e.g., Corning Ultra-Low attachment surface) to enable cell attachment to and retention within the scaffold, for instance at 37° C. in 5% CO₂ and humidified atmosphere. Cell attachment may be verified by fixing and staining cross-sections of the cell construct (e.g., staining the cell nuclei with DAPI (4′,6-diamidino-2-phenylindole) and analyzing it under fluorescence microscopy). Preferably, seeding efficiency is of at least 85%, 90%, 95%, 96%, 97%, 98%, 99% or 100%. In preferred embodiments, the human cell construct will be cultured for 2-4 hours, preferably around 3.5 hours.

In step (v) the cardiac construct is transferred to a perfusion bioreactor with electrostimulation capabilities to promote cardiomyocyte maturation. Perfusion of fresh oxygenated culture medium is conducted, preferably at a flow rate per chamber of 0.1 or 0.2 ml/min.

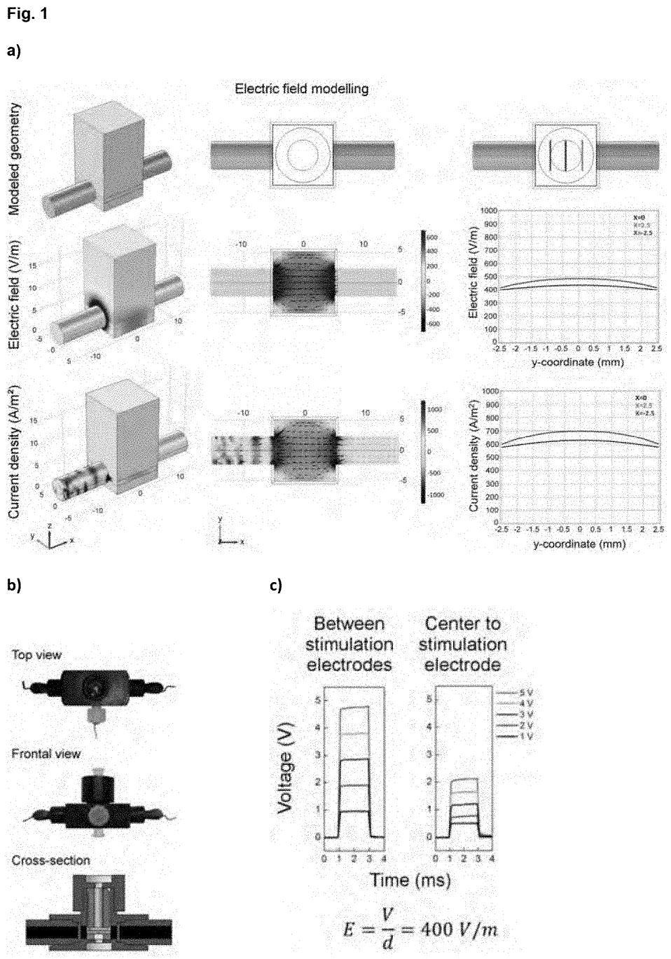

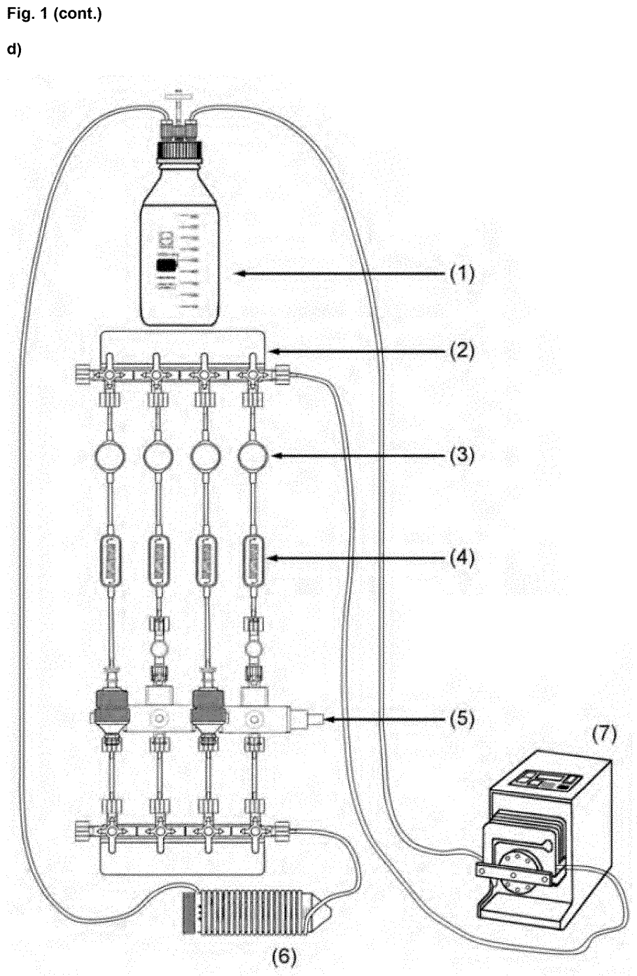

Preferably, electrical stimulation is applied in the culture chamber so that the cardiac construct is submitted to an electric field below 8 V/cm (Tandon et al. 2009), more preferably to an electric field around 400 V/m and a current density around 600 A/m². These correspond to the values of the parameters when measured in the center of the culture chamber. For instance, when using a culture chamber as shown in FIG. 1, electrical stimulation is obtained by the application of rectangular pulses at 1 Hz of frequency, of 2 minutes of duration at a differential voltage of 5 V when using two electrodes of about 5 mm separated about 1 cm.

Preferably, in step (v) the cardiac construct is cultured under perfusion for 3 days and electrical stimulation is also continuously applied from day 4 onwards. Preferably, cell constructs are cultured in step (v) for at least 7 days, more preferably for at least 14 days.

Electrical stimulation has been proposed to be a requirement for the electrophysiological maturation of cardiac constructs, as it is a well-known regulatory signal that favors cardiomyocytes contractility, alignment and organization within cardiac tissue constructs (Radisic, M. et al, 2004; Nunes, S. S. et al, 2013). As shown in the Examples, cardiac constructs were stimulated at a frequency of 1 Hz to mimic the electrical pacing in the native human adult heart, as it was previously shown that cardiomyocytes cultured in 3D aggregates adapted their autonomous beating rate to the frequency of stimulation (Eng, G. et al, 2016). After 14 days in culture, the cardiac constructs (also referred as “CardioSlice” constructs) displayed signs of increased tissue maturation compared to control constructs: cardiomyocytes aligned to one another following the direction of the electric field. At the ultrastructural level, electrical stimulation yielded improved myofilament structures, as evidenced by wider sarcomeres, and more developed intercellular unions than non-stimulated macrotissues. The alignment of cardiac cells and an increased myofibril ultrastructural organization has been connected to the improved electrical and mechanical properties of cardiac constructs (Fleischer, S. et al, 2017; Mathur, A. et al, 2015). Consistent with this, CardioSlice constructs showed improved electromechanical coupling which resulted in contractions with amplitude 6-fold higher than that of control constructs. Moreover, ECG-like signals elicited by CardioSlice constructs showed a uniform and reproducible pattern of narrow, steep and well-defined QRS complexes that very much resembled actual ECG heart recordings. In contrast, the bioelectrical signals generated by control constructs were highly heterogeneous, of comparatively lower amplitude and longer duration of waveforms, indicative of slowly conducting tissues.

Another aspect of the present disclosure refers to a human tridimensional macroscale cardiac construct obtained or obtainable by a method as described herein.

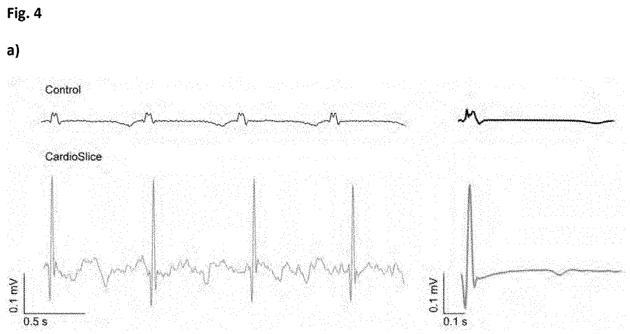

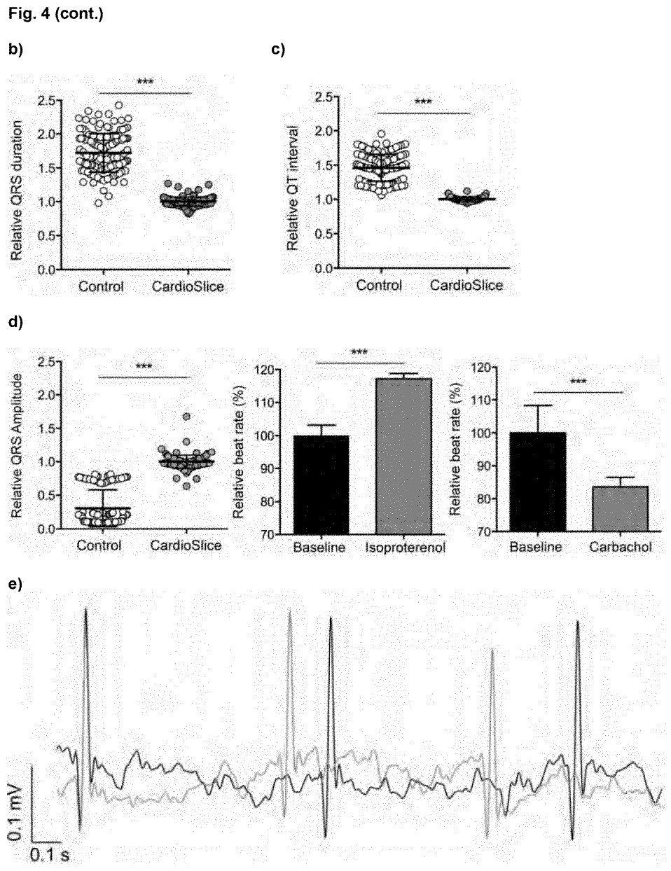

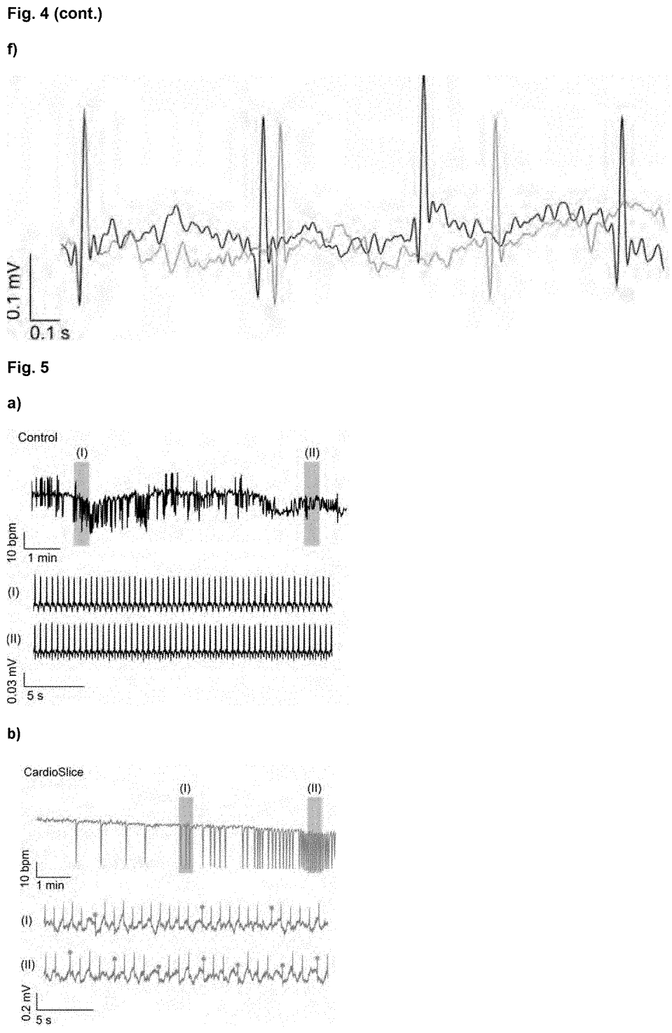

The cardiac construct obtained by the method as described herein resembles myocardial tissue both structurally and functionally (as demonstrated by the electrocardiogram (ECG) which accounts for improved synchronization and electrical signal propagation, see FIG. 4).

Accordingly, this cardiac construct may further be characterized by one or more, preferably all of the following features:

comprises mature cardiomyocytes expressing cardiac contractile proteins;

shows an increase in sarcomere width, and/or better development of intercalated discs with respect to control constructs (obtained in the absence of electrical stimulation);

presents aligned cells with synchronized beating (e.g., cells are aligned along the direction of electrical field applied and contracted in parallel to the direction of the electric field),

presents improved maturation of the electromechanical coupling machinery (as shown by increased amplitude of contraction with respect to control constructs obtained in the absence of electrical stimulation);

electrocardiogram graphs resemble those of healthy human myocardial tissue (e.g., the recorded electrical signals have narrow and step waveforms, and QRS complexes and repolarizing waves).

The mature cardiomyocytes, or a substantially pure population of mature cardiomyocytes, comprised in the cardiac cell construct obtained further to step (v) may also be characterized by expressing the markers cardiac Troponin T (cTnT) and/or alpha sarcomeric actin (ASA).

In one particular embodiment, the mature cardiomyocytes and/or the substantially pure population of mature cardiomyocytes express one or more of cTnT and ASA at a detectable level. In a further embodiment, the mature cardiomyocytes and/or the substantially pure population of mature cardiomyocytes express both cTnT and ASA at a detectable level.

In one particular embodiment, at least about 80%, 85%, 90%, 95%, 97% or 100% of the cardiomyocytes in the substantially pure cardiomyocytes population express cTnT and/or ASA at a detectable level. In another particular embodiment, at least about 70%, 75%, 80%, 85%, 90%. 95% or 100% of the cardiomyocytes in the substantially pure cardiomyocytes population express cTnT and ASA at a detectable level. In any of these embodiments, the indicated expression levels are for instance when expression is determined by flow cytometry or fluorescence-activated cell sorting (FACS) analysis.

In addition, the mature cardiomyocytes in the cardiac construct may also be characterized by being responsive to positive and negative inotropic factors. In particular, as described in the Examples, the obtained cardiac constructs have shown to modulate its beating rate upon treatment. For instance increasing its rate with a beta-adrenergic agonist compound and decreasing it upon treatment with a beta-adrenergic antagonist or a cholinergic agonist.

In preferred embodiments, the obtained macroscale cardiac construct has between 5 and 50 mm, preferably about 10 mm or about 20 mm in diameter; and between 0.5 and 3 mm, preferably between 1 and 2 mm, in thickness. Preferably, it has about 10 mm of diameter and thickness of about 1 mm.

In a further aspect, the disclosure refers to a cardiac construct as defined herein, for use in the treatment of a human subject having cardiac damage, for instance by replacement or regeneration of the damaged cardiac tissue. In a related aspect, the disclosure refers to a method of treating a subject having cardiac damage by administration of a therapeutically effective amount of the cardiac cell construct described herein. In a particular embodiment, of any thereof, said subject has ischemic heart disease. The term “ischemic heart disease” refers to a disease characterized by reduced blood supply to the heart. For instance, said subject has suffered a myocardial infarction or angina pectoris event.

The term “effective amount” as used herein refers to an amount that is effective, upon single or multiple dose administration to a subject (such as a human patient) in the prophylactic and/or therapeutic treatment of a disease, disorder or pathological condition.

In an additional aspect, the present disclosure refers to the in vitro use of a cardiac construct as described herein, for the screening or evaluation of compounds, such as drugs, on cardioprotective or cardiotoxic properties. In another aspect, the present disclosure refers to the in vitro use of a cardiac construct as described herein, for cardiac disease modeling.

In still another aspect of the present disclosure refers to a perfusion bioreactor with electrical stimulation capabilities, which would thus be suitable for electrostimulating cardiac cells with the desired pulsatile electric field.

A perfusion cell culture process involves the constant feeding of fresh media and removal of spent media and product while retaining high numbers of viable cells. Continuous perfusion of fresh media may be achieved through the use of a peristaltic pump. In a parallelized bioreactor, media can be equally distributed through the various (e.g., four) branches of the bioreactor by using flow restrictors. In some embodiments, the bioreactor is a closed-circuit and elimination of waste products is achieved by changing the culture medium manually, preferably every day, using sterile syringes. Since cells are attached to the scaffold, they are not affected by culture medium changes.

In a particular embodiment, the bioreactor comprises at least one perfusion chamber and two electrically stimulating electrodes within the chamber. Preferably, said bioreactor is a parallelized bioreactor with multiple culture chambers, for instance with 2, 3, 4, 5 or 6 chambers capable to simultaneously provide fluid perfusion and electrical stimulation to several cardiac constructs. Thus, allowing the production of multiple cardiac macrotissues under the same physiochemical conditions. The stimulating electrodes are preferably made of graphite. The bioreactor may further comprise a measuring electrode, preferably made of gold, connected with the chamber to be used as internal reference to take measurements in the center of the perfusion chamber. In a preferred embodiment, this parallelized perfusion bioreactor with electrical stimulation and measurement capabilities are as defined in FIG. 1.

By incorporating electrodes to the bioreactor chamber, the bioreactor is provided with means to stimulate the cell constructs. In addition, the bioreactor is provided with the capability of on-line monitoring the electrophysiological behavior of the constructs in a non-destructive manner. Accordingly, in a further aspect, the disclosure provides a method for the non-destructive evaluation of electrophysiological activity in a cardiac construct, said method comprising the use of a bioreactor as described herein enabling the registration of electric signals, such as ECG-like signals.

Since no standard method to assess electrophysiological information from intact macroscale-sized heart tissue exists (Tzatzalos, E. et al, 2016), the technology developed here is unique in this context. Electromechanical coupling is usually evaluated through contractility measurements under a microscope (Radisic, M. et al, 2004; Nunes, S. S. et al, 2013; Hirt, M. N. et al, 2014). Electrophysiological activity, in turn, is recorded on isolated cardiomyocytes after tissue formation (Schaaf, S. et al, 2011; Nunes, S. S. et al, 2013), therefore requiring the destruction of the sample. Electrical activity of disaggregated cells is obtained by measuring transmembrane action potentials (Liang, P. et al, 2013), microelectrode array (MEA) recordings (Pradhapan, P. et al, 2013), impedance measurements (Nguemo, F. et al, 2012), and calcium- (Fleischer, S. et al, 2017) or voltage- (Yan, P. et al, 2102) sensitive dyes. Through the novel set-up described herein, real-time monitoring of the electrophysiological activity in thick human cardiac macroscale tissue-like constructs has been demonstrated, and ECG-like signals registered.

It is contemplated that any features described herein for the human cardiac construct can optionally be combined with any of the embodiments of any method of production, any medical use, method of treatment, method for the screening or evaluation of compounds, method for cardiac disease modelling, bioreactor or method for the non-destructive evaluation of electrophysiological activity in a cardiac construct of the invention; and any embodiment discussed in this specification can be implemented with respect to any of these. It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications are herein incorporated by reference to the same extent as if each individual publication or patent application was specifically and individually indicated to be incorporated by reference.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed disclosure. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, 10, 11, 12, 13, 14 or 15%. Accordingly, the term “about” may mean the indicated value±5% of its value, preferably the indicated value±2% of its value, most preferably the term “about” means exactly the indicated value (±0%).

The following examples serve to illustrate the present disclosure and should not be construed as limiting the scope thereof.

### EXAMPLES

### Example 1

### Material and Methods

### Parallelized Perfusion Bioreactor System

Parallelized perfusion bioreactor (FIG. 1d) was composed of a medium reservoir (Sartorius Stedim Biotech) connected through gas-permeable platinum-cured silicone tubing (1.6 mm inner diameter, Thermo Fisher Scientific) to a PharMed® BPT 3-Stop pump tubing (0.89 mm ID, Cole Parmer). The pump tubing was connected to a multichannel peristaltic pump (REGLO Digital, 2 channels, Ismatec) that propelled the culture medium to a four-port luer manifold (Thermo Fisher Scientific). In each port of the luer manifold, the infusion line of the elastomeric infusion system DOSI-FUSER® was connected, composed of an air and particle filter (1.2 μm particle filter and 0.02 μm vent filter) and a flow restrictor (1·10⁻² cm inner diameter and 10.8 cm length) (L25915-250D2, Leventon). The flow restrictor allowed an equal distribution of the culture medium throughout the four branches of the bioreactor, and the filter acted as a high fidelity de-bubbling system to avoid entrapment of bubbles inside perfusion chambers. An in-line luer injection port (Inycom) was assembled before the perfusion chambers to allow direct drug injection. Finally, the four branches were assembled with another luer manifold attached to a gas exchanger, composed of 3 m of gas-permeable platinum-cured silicone tubing (1.6 mm inner diameter, Thermo Fisher Scientific) coiled around a holder. To close the circuit, the gas exchanger was connected to the medium reservoir. All connections between the components were performed using male and female luer lock connectors (for 1.6 mm tube inner diameter, Value Plastics). Two different perfusion chambers were used to either electrically stimulate cardiac constructs or not. The perfusion chamber without electrodes was a Swinnex filter holder (13 mm, Merck Millipore), while the perfusion chamber that enabled electrical stimulation was designed and fabricated in-house (See “Fabrication and characterization of the perfusion chamber with electrical stimulation” as described below).

Notably, both chambers had equivalent inner dimensions to obtain comparable tissue constructs. In both chambers the cardiac construct was held in place by two gaskets, and a continuous perfusion of culture medium at 0.1 ml/min per chamber was applied (0.4 ml/min total flow for a parallel system allocating 4 chambers). For electrically stimulated cardiac constructs, trains of monophasic square-wave pulses of 2 ms of duration and 5 V of amplitude (peak to peak) were continuously applied from day 4 of culture until the end of the experiment. For human CardioSlice constructs the frequency of the pulses was of 1 Hz, while for rat cardiac constructs it was of 3 Hz. Control cardiac constructs were cultured under the same flow conditions but without electrical stimulation. All the components were sterilized by either autoclaving or 70% ethanol with subsequent MilliQ water rinse. The whole system was placed inside an incubator with temperature and CO₂ control (37° C. and 5% CO₂). Images and diagrams of the bioreactor and culture chambers were processed using GNU Image Manipulation Program (The GIMP team, GIMP 2.8.18, www.gimp.org, 1997-2016).

Fabrication and Characterization of the Perfusion Chamber with Electrical Stimulation

Our custom-made perfusion chamber with electrical stimulation was fabricated by precision machining of polypropylene (PP) plastic, followed by gluing of luer connectors using cyanoacrylate. To achieve a completely watertight chamber, silicone O-rings (4.6 mm inner diameter, The O-Ring Store, LLC) and thread seal tape was used. The perfusion chamber had an inlet and an outlet to allow culture medium perfusion, two carbon rod electrodes of 3/16″ in diameter (Monocomp Instrumentación) to electrically stimulate cells and one gold electrode of 0.5 mm in diameter (Advent Research Materials) as a measuring electrode (FIG. 1b). Two holes were drilled at one edge of each rod electrode, and a solid tinned annealed copper wire (RS Pro) was thread through the holes. Insulation of the connection was performed using Araldite® epoxy resin, and waterproofing of rod electrodes was achieved using heat-shrink tubing (Thermo Fisher Scientific). Electric potential values between stimulation electrodes and between one stimulation electrode and the center of the chamber (gold electrode) were characterized using a function generator (Agilent Technologies) and an oscilloscope (Agilent Technologies) (FIG. 1c).

### Electric Field Modeling

COMSOL Multiphysics® software was used to predict the electric field and the current density that stimulates cells in our custom-made perfusion chamber. The electric current module was used, which considers the conductivity and permittivity of each material to solve a current conservation problem for a given electric potential. Electric fields throughout our geometry were calculated by assuming steady state, as previously described (Tandon, N. et al, 2009; Barash, Y. et al, 2005; Tandon, N. et al, 2011). To run the simulation, the exact geometry of our perfusion chamber was designed except for its internal part, where a prism was drawn to faithfully reproduce the interaction between the electrodes and the culture medium (FIG. 1a). The model was solved for a mesh with an average element size of 0.0473 mm² by applying a differential potential of 5 V between the stimulating electrodes (graphite rods, 3/16″ in diameter, Monocomp Instrumentación). The conductivity value of the cell culture medium used for the simulation was 1.44±0.03 S/m, measured with a conductivity meter (Crison) using DMEM 4.5 g/l glucose (Life Technologies).

### Structural and Mechanical Analysis of the Scaffold

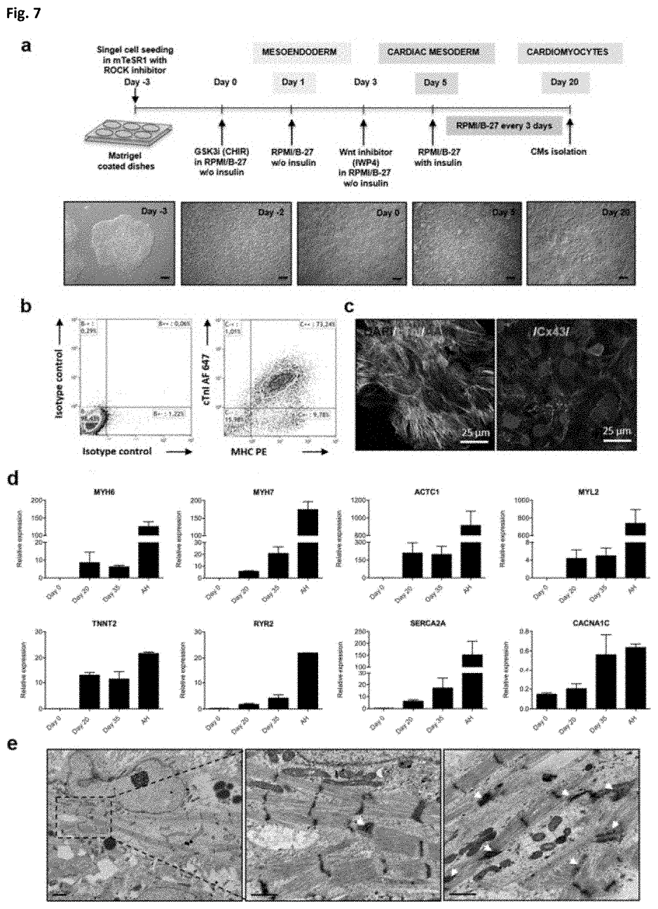

Commercially available collagen and elastin-based sponges (Matriderm®, MedSkin Solutions Dr. Suwelack A G) were used as 3D scaffolds (FIG. 7). Scaffold morphology and mean pore size was analyzed in dry conditions by scanning electron microscopy (SEM) (Nova NanoSEM™ 230, FEI) using the low vacuum mode (1 mbar water pressure) without any conductive coating. Second Harmonic Generation (SHG) and two-photon excited fluorescence (TPEF, autofluorescence) using an inverted confocal microscope (Leica SP5, Leica Microsystems) was used to elucidate scaffold morphology in hydrated conditions (Richards-Kortum, R. & Sevick-Muraca, E., 1996; Jiang, X. et al, 2011). Images were analyzed using ImageJ free software (National Institutes of Health, USA). The stiffness of the hydrated scaffold (disks of 10 mm in diameter) was measured in compression using a Q800 Dynamic Mechanical Analyzer (TA instruments). A ramp strain of −0.5%/min rate to a maximum strain of −5% and a preload force of 0.01 N were applied. Young Modulus (E) of the scaffold was determined from the slope of the linear stress-strain curves at both room temperature and at 37° C.

Human iPSC Culture and Cardiac Differentiation

Human induced pluripotent stem cells (hiPSC) (FiPS Ctrl1-mR5F-6; cell line registered in the National Stem Cell Bank, Institute of Health Carlos III, Spanish Ministry) were cultured on Matrigel® (10 cm diameter, Corning) coated dishes with mTeSR1 medium (Stem Cell Technologies). Cells were differentiated into cardiomyocytes in monolayer culture with modulators of canonical Wnt signaling as described in further detail in the section below and in FIG. 7a. Briefly, monolayer cultures on Matrigel® in a serum-free medium were treated with 10 μM GSK3 inhibitor (CHIR99021, Stemgent) for 24 h (day 0 to day 1). On day 3 of differentiation, cells were treated with 5 μM Wnt inhibitor IWP4 (Stemgent) for 2 days. Contracting cardiomyocytes were obtained between 8 and 12 days of differentiation. Beating clusters were disaggregated (at day 20 and at day 35) by incubation with 0.25% trypsin-EDTA (Gibco) for 5-8 min at 37° C., both for their characterization and for cardiac constructs generation. Images were processed using ImageJ free software (National Institutes of Health, USA).

Human iPSC Culture and Cardiac Differentiation

Human iPSC were cultured on 10 cm Matrigel (Corning) coated dishes with mTeSR1 medium (Stem Cell Technologies). Medium was changed every day, excluding the day right after passaging. Cells were split 1:6-1:10 by incubation with 0.5 mM EDTA (Invitrogen) for 2 min at 37° C. and cell aggregates were plated on Matrigel coated dishes and maintained in culture for subsequent passages. Human iPSC were differentiated into cardiomyocytes in monolayer culture with modulators of canonical Wnt signaling as previously described⁴⁶. Cells maintained on Matrigel in mTeSR1 medium were dissociated into single cells with Accutase (Labclinics) at 37° C. for 8 min and seeded onto Matrigel-coated 12-well plate at a density of 1.5 million cells per well in mTeSR1 medium supplemented with 10 μM ROCK inhibitor (Sigma). Cells were cultured in mTeSR1 medium, changed daily during 3 days. When human iPSC achieved confluence, cells were treated with 10 μM GSK3 inhibitor (CHIR99021, Stemgent) in RPMI (Invitrogen) supplemented with B27 lacking insulin (Life Technologies), 1% glutamax (Gibco), 0.5% penicilin-streptomycin (Gibco), 1% non-essential amino acids (Lonza), and 0.1 mM 2-mercaptoethanol (Gibco) (RPMI/B27-insulin medium) for 24 h (day 0 to day 1). After 24 h, the medium was changed to RPMI/B27-insulin and cultured for another 2 days. On day 3 of differentiation, cells were treated with 5 μM Wnt inhibitor IWP4 (Stemgent) in RPMI/B27-insulin medium and cultured without medium change for 2 days. Cells were maintained in RPMI supplemented with B27 (Life Technologies), 1% L-glutamine, 0.5% penicilin-streptomycin, 1% non-essential amino acids, and 0.1 mM 2-mercaptoethanol (RPMI/B27 medium) starting from day 7, with medium change every 2 days. On day 8, contracting cardiomyocytes were obtained. Beating clusters were disaggregated (at day 20 and at day 35) by incubation with 0.25% trypsin-EDTA (Gibco) for 5-8 min at 37° C., both for their characterization and in vitro studies.

### Isolation and Culture of Neonatal Rat Cardiomyocytes

Hearts from 2-3-day-old Sprague-Dawley rats were isolated following a protocol approved by Animal Experimentation Ethics Committee of the University of Barcelona (Barcelona, Spain). Briefly, ventricular tissue was excised, cut into two parts and washed with cold Calcium and Bicarbonate-Free Hank's Balanced Salt Solution with HEPES (CBFHH) buffer. Then, ventricles were cut sharply into small pieces (<1 mm³) and subjected to 20-25 cycles (3 min each, room temperature) of enzymatic digestion using ice-cold 2 mg/ml trypsin (BD Difco™) in CBFHH and ice-cold 4 μg/ml DNAse I (Calbiochem, Merck Millipore) in CBFHH. Pooled supernatants were collected and centrifuged at 100×g for 12 min, and the pellet was resuspended in cold DMEM containing 1 g/l glucose (Life Technologies) supplemented with 10% FBS, 100 μM nonessential amino acids (Life Technologies), 2 mM L-glutamine (Life Technologies), 50 U/ml penicillin and 50 μg/ml streptomycin (Life Technologies). Cell suspension was filtered through a 250 μm stainless steel test sieve (Filtra Vibración), seeded into Matriderm® scaffolds or in 12-well plates and cultured in DMEM containing 4.5 g/l glucose (Life Technologies) supplemented with 10% horse serum (Life Technologies), 2% Chick Embryo Extract (EGG Tech), 100 μM nonessential amino acids, 2 mM L-glutamine, 50 U/ml penicillin and 50 μg/ml streptomycin.

### Generation of Cardiac Macrotissues

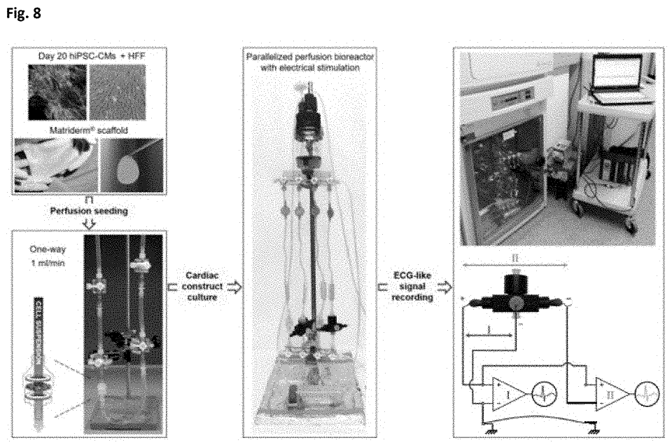

FIG. 8 provides the reader with a summary of the strategy followed to generate cardiac macrotissues. A porous collagen and elastin-based scaffold of 1 mm thickness (Matriderm®) was used. The commercial scaffold was cut in disks of 10 mm in diameter and hydrated in phosphate buffered saline (PBS) (Thermo Fisher Scientific) for 24 hours before use. For human cardiac constructs, 4.5·10⁶ human iPSC-derived cardiomyocytes selected at day 20 of differentiation and 0.5·10⁶ human foreskin fibroblasts (HFF) resuspended in 1 ml of RPMI (Invitrogen) supplemented with B27 (Life Technologies) medium were seeded in each scaffold. For rat cardiac constructs, 3.5·10⁶ cells isolated from rat heart ventricles resuspended in 1 ml of supplemented DMEM were used. Each cell suspension was seeded into the scaffold using an adapted version of a perfusion loop system (Radisic, M. et al 2008) (FIG. 8). Briefly, the cell suspension was loaded inside the loop and a flow rate of 1 ml/min was applied in one direction, forcing the cell suspension to pass through the scaffold. After seeding, tissue constructs were placed in 60 mm ultra low attachment dishes (Corning) and incubated at 37° C. in 5% CO₂ and humidified atmosphere to allow cell attachment. Human cardiac constructs were incubated for 3.5 hours, while rat cardiac constructs were incubated for 1.5 hours. Then, tissue constructs were transferred into the bioreactor perfusion chambers and cultured under perfusion for 3 days. At day 4 of culture, electric field stimulation regimen was applied on CardioSlice tissue constructs, while control ones were cultured only under perfusion. All tissue constructs were cultured for either 7 or 14 days before assessing their functional and structural organization level. As 2D controls of cardiac macrotissues, day 20-differentiated cardiomyocytes together with 10% HFF or cells isolated from rat heart ventricles were seeded on 0.1% gelatin-coated 12-well plates with 12 mm coverslips (Thermo Scientific) at a density of 2·10⁵ cells/cm². Images and diagrams of the perfusion loop system were processed using GNU Image Manipulation Program (The GIMP team, GIMP 2.8.18, www.gimp.org, 1997-2016).

### Immunostaining Analysis

Scaffolds were fixed overnight with 4% paraformaldehyde (PFA, Sigma) at 4° C. and included in 8% agarose (Conda) for 5 min at 4° C. Blocks were cut in 200 μm sections using a vibratome (0.075 mm/s advance rate, 81 Hz vibration and 1 mm amplitude) (Leica VT1000S). After three washes in 1×TBS (30 min each), sections were incubated in blocking solution I (1×TBS, 0.5% Triton-X100 (Sigma) and 6% donkey serum (Chemicon)) for 2 h at room temperature, and incubated with primary antibodies diluted in blocking solution II (1×TBS, 0.1% Triton-X100 and 6% donkey serum) for 72 h at 4° C. in agitation. After four washes in 1×TBS (30 min each), sections were incubated with secondary antibodies diluted in blocking solution II for 2 h in the dark and overnight at 4° C. Finally, sections were washed thrice with 1×TBS for 30 min each and incubated with DAPI (Invitrogen) for 1 h at room temperature. Rat neonatal and differentiated cardiomyocytes seeded on gelatin-coated coverslips were fixed with 4% PFA for 15 min at room temperature and used as 2D controls. Images were taken using a SP5 confocal microscope (Leica Microsystems) and analyzed using ImageJ free software (National Institutes of Health, USA). Primary and secondary antibodies used are listed in Table 1.

TABLE 1
List of the primary and secondary antibodies used for immunohistochemistry analysis
They were used to characterize both 2D cell cultures and cardiac macrotissues.

| Type | Antibody | Host | Dilution | Manufacturer |
|---|---|---|---|---|
| Primary | Actin α-sarcomeric (Monoclonal IgM) | Mouse | 1:400 | Sigma-Aldrich, A2172 |
| Primary | α-actinin sarcomeric (Monoclonal IgG1) | Mouse | 1:100 | Sigma-Aldrich, A7811 |
| Primary | Connexin-43 (Polyclonal IgG) | Rabbit | 1:250 | Abcam, ab11370 |
| Primary | Troponin I (Polyclonal IgG) | Rabbit | 1:25 | Santa Cruz, sc-15368 |
| Primary | Troponin T (Monoclonal IgG1) | Mouse | 1:100 | Thermo Fisher, MS-295-P |
| — | Phalloidin, Texas Red ® | — | 1:40 | Life technologies, T7471 |
| Secondary | Alexa Fluor ® 488, Goat IgG | Donkey | 1:200 | Jackson, 705-545-147 |
| Secondary | Alexa Fluor ® 488, Mouse IgG | Goat | 1:200 | Jackson, 115-546-071 |
| Secondary | Alexa Fluor ® 488, Mouse IgG | Donkey | 1:200 | Jackson, 715-545-151 |
| Secondary | Cy ™3, Mouse IgG | Goat | 1:200 | Jackson, 115-165-071 |
| Secondary | Cy ™5, Mouse IgG | Goat | 1:200 | Jackson, 115-175-071 |
| Secondary | Cy ™2, Mouse IgM | Goat | 1:200 | Jackson, 115-225-075 |
| Secondary | Cy ™3, Mouse IgM | Donkey | 1:200 | Jackson, 715-165-140 |
| Secondary | Cy ™3, Mouse IgM | Goat | 1:200 | Jackson, 115-165-075 |
| Secondary | DyLight ™ 649, Mouse IgM | Goat | 1:200 | Jackson, 115-495-075 |
| Secondary | Cy ™5, Mouse IgM | Rabbit | 1:200 | Jackson, 315-175-049 |
| Secondary | Alexa Fluor ® 488, Rabbit IgG | Donkey | 1:200 | Jackson, 711-545-152 |
| Secondary | Cy3, Rabbit IgG | Donkey | 1:200 | Jackson, 711-165-152 |

### Flow Cytometry Analysis

Characterization of human iPSC-derived cardiomyocytes was performed by flow cytometry analysis. Cells were dissociated on day 19 of differentiation using 0.25% trypsin-EDTA at 37° C. for 5 min and then fixed with 4% paraformaldehyde (Sigma) for 20 min at room temperature. After washing with 1× saponin (Sigma), cells were permeabilized using Cell Permeabilization Kit (Invitrogen) and blocked with 5% mouse serum during 15 min at room temperature. Then, cells were stained with the antibodies mouse PE-anti myosin heavy chain (MHC) (IgG2b, 1:400 BD Biosciences) and mouse Alexa Fluor 647 cardiac troponin I (cTnI) (IgG2b, 1:100, BD Biosciences). Mouse IgG2b PE (1:400 BD Biosciences) and mouse IgG2b Alexa Fluor 647 (1:100, BD Biosciences) antibodies were used as isotype controls. After incubation during 15 min at room temperature in the dark and washing twice with 1× saponin, cells were analyzed with FACS MoFlo (Beckman Coulter) and data acquisition and analysis performed by Kaluza software (Beckman Coulter).

### Functional Analysis of Cardiac Macrotissues

Contractile function of cardiac macrotissues in spontaneous beating and in response to electric field stimulation was assessed in a set-up equivalent to one previously described (Tandon, N. et al, 2009; Tandon, N. et al, 2011). Briefly, two holes were drilled at one edge of two carbon rod electrodes, and a gold wire of 0.5 mm in diameter was thread through them. Insulation of the connection was performed using heat-shrink tubing (Thermo Fisher Scientific), and both electrodes were glued using cyanoacrylate at the bottom of a 35 mm MatTek glass bottom dish (MatTek In Vitro Life Science Laboratories), 1 cm apart from the edge of each electrode. The space between the electrodes was filled with Tyrode's salts solution (Sigma-Aldrich Quimica), and cardiac macrotissues were imaged and video recorded using a Stereo Microscope Leica MZ10F (Leica Microsystems) with a DFC4025C Digital Microscope Camera (Leica Microsystems). Temperature was maintained at 37° C. using a microscope-stage automatic thermocontrol system for transmitted light bases (Leica MATS Type TL, Leica Microsystems), and electrical pulses were applied using a function generator (33250A, Agilent Technologies). To determine the maximum capture rate (MCR) of the constructs, square-wave pulses of 2 ms of duration, 10 V of peak-to-peak amplitude (Vₚₚ) and 1 Hz of frequency were applied, and frequency was increased in 0.1 Hz until the cardiac macrotissue was no longer synchronously beating at the paced frequency. To measure the amplitude of contraction of each cardiac macrotissue, spontaneous beating of the constructs was recorded for at least 10 s. Then, the Fractional Area Change (FAC) of 10 beats was calculated using a custom MATLAB program (v. R2014b, Natick, Mass., USA) as previously described (Tandon, N. et al., 2009). Particle Image Velocimetry (PIV) was used to measure the velocity fields and the strain rate in beating cardiac tissue constructs. Recorded videos were analyzed using the PIVlab 1.41 software package for MATLAB (v. R2014b, Natick, Mass., USA) (Thielicke, W. & Stamhuis, E. J., 2014a; Thielicke, W. & Stamhuis, E. J., 2014b; Thielicke, W., 2014). Each frame was cross-correlated with the preceding one using 77 μm×77 μm interrogation windows, obtaining local displacement fields (velocity field). The alignment between velocity vector fields and the direction of the electric field was assessed by the order parameter <cos 2θ> in every frame. If vectors were aligned in the direction of electric field, <cos 2θ> value was 1, whereas if vectors were perpendicular to the electric field, its value was −1. Random distribution was represented by a <cos 2θ> value close to 0. To determine the strain rate, built-in PIVlab functions were used. An integral of the strain rate over the beating area was determined for each frame. Positive strain rates were added up and divided by the number of contractions to obtain the strain per contraction. Images of cardiac macrotissues were taken from each video, and processed using GNU Image Manipulation Program (The GIMP team, GIMP 2.8.18, www.gimp.org, 1997-2016).

### Quantitative Real-Time Polymerase Chain Reaction

Total RNA was isolated from differentiated cells and cardiac macrotissues using Trizol RNA Isolation Reagent (Life Technologies), and 1 μg was used to synthesize cDNA using Transcriptor first-strand cDNA synthesis kit (Roche) according to the manufacturer's protocol. The quantitative real-time polymerase chain reaction (qRT-PCR) was carried out using the 7900 HT Fast Real-Time PCR System (Applied Biosystems). Human GAPDH was used a housekeeping gene. RNA from human fetal and adult heart (Clontech) was used as positive control. Specific primers are listed in Table 2.

TABLE 2
Quantitative real-time polymerase
chain reaction primers

| Gene | Sense | Antisense |
|---|---|---|
| MYH6 | ATTGCTGAAACCGAGAATGG | CGCTCCTTGAGGTTGAAAAG |
| | (SEQ ID NO: 1) | (SEQ ID NO: 2) |
| MYH7 | GCATCATGGACCTGGAGAAT | ATCCTTGCGTTGAGAGCATT |
| | (SEQ ID NO: 3) | (SEQ ID NO: 4) |
| ACTC1 | GCTCTGGGCTGGTGAAGG | TTCTGACCCATACCCACCAT |
| | (SEQ ID NO: 5) | (SEQ ID NO: 6) |
| TNNT2 | TGCAGGAGAAGTTCAAGCAG | AGCGAGGAGCAGATCTTTGG |
| | CAGA (SEQ ID NO: 7) | TGAA (SEQ ID NO: 8) |
| MYL2 | CAACGTGTTCTCCATGTTCG | GTCAATGAAGCCATCCCTGT |
| | (SEQ ID NO: 9) | (SEQ ID NO: 10) |
| GATA4 | GCGGCCTCTACATGAAGCTC | CTTCCGTTTTCTGGTTTGGA |
| | (SEQ ID NO: 11) | (SEQ ID NO: 12) |
| GJA1 | CAATCACTTGGCGTGACTTC | CCTCCAGCAGTTGAGTAGGC |
| | (SEQ ID NO: 13) | (SEQ ID NO: 14) |
| RYR2 | ACAGCACAAGCCATTCTGCA | ATGTAATCCAGCCCACCCAG |
| | AGA (SEQ ID NO: 15) | ACAT (SEQ ID NO: 16) |
| SERCA2A | TGAGACGCTCAAGTTTGTGG | TCATGCACAGGGTTGGTAGA |
| | (SEQ ID NO: 17) | (SEQ ID NO: 18) |
| CACNA1C | TTTGGTCCATGGTCAATGAG | GCATTGGCATTCATGTTGG |
| | (SEQ ID NO: 19) | (SEQ ID NO: 20) |
| SCN5A | GGGCAATGTCTCAGCCTTAC | CATCAGCCAGCTTCTTCACA |
| | (SEQ ID NO: 21) | (SEQ ID NO: 22) |
| KCNH2 | CCTTCGACCTGCTCATCTTC | TGAGTAGCGATCCAGCTTCC |
| | (SEQ ID NO: 23) | (SEQ ID NO: 24) |
| KCNQ1 | CGCCTGAACCGAGTAGAAGA | AAGGAGAGCAGCTGGTGAAG |
| | (SEQ ID NO: 25) | (SEQ ID NO: 26) |
| GAPDH | AGGGATCTCGCTCCTGGAA | AGGGATCTCGCTCCTGGAA |
| | (SEQ ID NO: 27) | (SEQ ID NO: 28) |

### Transmission Electron Microscopy

Cardiac macrotissues were fixed with 2.5% glutaraldehyde (Electron Microscopy Sciences) for 2 h at 4° C. After washing with 0.1 M cacodylate buffer (pH=7.2) (Sigma-Aldrich), cardiac macrotissues were gradually dehydrated with ethanol and embedded in epoxy resin (Ted Pella). Semithin sections (0.25 μm) were cut with a diamond knife using an ultramicrotome (Leica UC6), and stained lightly with 1% toluidine blue (Panreac). Later, ultra-thin sections (0.08 μm) were cut with a diamond knife, contrasted with uranyl acetate (Electron Microscopy Sciences) and lead citrate (Electron Microscopy Sciences) and examined under a JEOL 1011 transmission electronic microscope (JEOL). Images were analyzed using ImageJ free software (National Institutes of Health, USA).

### Drug Treatment

Isoproterenol, carbachol and sotalol were purchased from Sigma-Aldrich. All chemicals were dissolved in distilled water to make stock solutions, and serial dilutions were made in RPMI (Invitrogen) culture medium. To test changes in the β-adrenergic response, 1 μM isoproterenol was injected to the bioreactor circuit through the luer injection port (Inycom). For cholinergic stimulation, 10 μM carbachol was injected, and sotalol was added at 10 μM to evaluate the blockade of hERG current (Braam, S. R. et al, 2013). Spontaneous activity of cardiac constructs was recorded as baseline period. Cardiac constructs were treated with pharmacological agents during 10 min for immediate analysis, followed by 5 min washout for isoproterenol and carbachol, and 15 min for sotalol.

### Recording and Processing of Electrocardiogram (ECG)-Like Signals Generated by Cardiac Macrotissues

Electrocardiogram (ECG)-like signals were acquired through the three electrodes configuration built in the custom-made cell culture chamber and using an advanced transducer amplifier. The gold electrode acted as internal reference, and electrical activity of cardiac macrotissues was acquired using the gold and graphite electrodes. In particular, “Amplifier I” acquires activity between: “Graphite+ and Reference”; and “Amplifier II” acquires activity between: “Graphite+ and Graphite−” (FIG. 8). The obtained ECG-like signal is generated by the sum of the synchronized action potentials of all cardiomyocytes. Its shape is similar to regular surface ECG signals for humans, including the presence of the QRS complex and T wave (FIG. 4a). The biomedical instrumentation for recording the bioelectrical signals was a MP150 system with a 16-Bits AD converter, and DAC100C amplifiers with gain 50, maximum bandwidth DC-5 kHz, input impedance 2 MΩ and CMRR≥90 dB (BIOPAC, Santa Barbara, Calif., USA). ECG-like signal recordings were monitored and stored in a computer using the AcqKnowledge software v.4.1 (BIOPAC, Santa Barbara, Calif., USA), and were analyzed with MATLAB (v. R2014b, Natick, Mass., USA). ECG-like signals were recorded at a sampling frequency of 12.5 kHz, and were filtered and decimated at a sampling rate of 500 Hz.

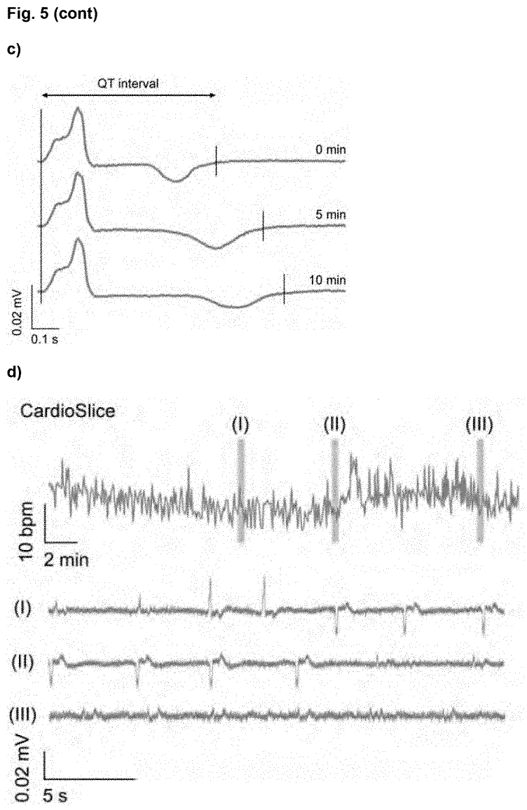

Recorded ECG-like signals were bandpass filtered (zero-phase fourth-order Butterworth filter with cut-off frequencies of 0.2 and 125 Hz), to use the main bandwidth of the classical electrocardiographic studies. Previously, the 50 Hz line interference and harmonics were cancelled by a comb-notch filter. To improve identification of patterns in the ECG-like signals (FIG. 4a), Ensemble Empirical Mode Decomposition (Wu, Z. H. & Huang, N. E. 2009) (Noise level: 1 dB; Iterations: 200) was used to cancel the multicomponent noise. Beat-to-beat fiducial points were detected and then signal averaging techniques were applied to estimate the main pattern of each ECG-like signal, enabling analysis and interpretation of QRS complex, T wave and QT interval (FIG. 4a and FIG. 5c).

During the experiments involving drug treatment, instantaneous beat rate expressed as beats per minute (bpm) was calculated over ECG-like signals, generated in both control and CardioSlice constructs at baseline and after drug application. Beat rate was assessed by measuring the RR interval, which is the time elapsing between two consecutive R waves in the electrocardiogram. Relative beat rate to baseline was calculated to evaluate the behavior of cardioactive drug effects.

### Statistical Analysis

Normal distribution of data was tested using the Shapiro-Wilk and Kolmogorov-Smirnov normality tests. Differences between Young modulus (E) and fractional area change (FAC) means were analyzed using Student's t-Test. Non-parametric analysis was performed using Mann-Whitney U test for sarcomere width, maximum capture rate (MCR) and <cos 2θ>, as well as to evaluate the beating rate change after isoproterenol and carbachol treatments. Regarding the average strain per contraction, means were compared using one-way ANOVA and subsequently analyzed using Tukey's post hoc test. The difference between the variance of experimental groups in QRS duration, QT interval and QRS amplitude was analyzed using two-sample test for variances (F-test). Software used were Origin 8.5 (OriginLab, Northampton, USA) and GraphPad Prism 6, and differences were considered significant when p<0.05.

### Results

Development of a Parallelized Bioreactor for Continuous Perfusion and Electrical Stimulation/Recording of Cardiac Macroscale Constructs

To generate CardioSlice constructs, a parallelized perfusion bioreactor including electrical stimulation was designed (FIG. 1). Our a priori design requirements included: (i) suitability for the perfusion of macroscale constructs (10 mm in diameter and 1-2 mm in thickness); (ii) capability for in vivo-like electrical stimulation; (iii) ability to produce interstitial flow with proper shear; (iv) amenability to parallelization for the simultaneous culture of multiple constructs; and (v) on-line recording of the construct electrical activity. To address these requirements, we first formulated an electric field model to determine the appropriate size and allocation of the stimulating electrodes within the chamber (FIG. 1a). We simulated the electric field and current density generated by two graphite electrodes (5 mm in diameter) separated 1 cm apart and in contact with culture medium. Solving the model resulted in an electric field around 400 V/m and a current density around 600 A/m² in the center of the chamber, when a differential voltage of 5 V was applied. Similar values were obtained for the entire region intended to use to allocate the cardiac construct containing cells, thus being this configuration suitable for cardiomyocyte stimulation in terms of magnitude and uniformity according to literature (Tandon, N. et al, 2009; Barash, Y. et al, 2010). We then fabricated custom-made perfusion chambers including electrodes with the specifications included in the simulation (FIG. 1b). Upon fabrication, we measured the voltage values in the actual perfusion chamber to verify that the expected stimuli were properly delivered (FIG. 1c). Rectangular pulses at 3 Hz of frequency, 2 ms of duration and increasing differential voltages from 1 to 5 V were applied to the chamber. Electric potentials between stimulating graphite electrodes and between each electrode and the center of the chamber (gold electrode) were measured. The intended waveform was reliably applied for all voltages tested. Moreover, when applying 5 V between graphite electrodes, 2 V were measured in the center of the chamber, representing an electric field of 400 V/m, in agreement with the values predicted by the model.

The shear stress to which cells would be exposed in the perfusion bioreactor was calculated as previously described (Radisic, M. et al, 2004). The minimum flow rate needed for efficiently perfusing cardiac macrotissues depends on the overall mass balance of oxygen, and it was estimated to be 0.1 ml/min for rat cardiac tissue constructs, and 0.2 ml/min for human cardiac tissues. Then, shear stress affecting the cells within the scaffold was calculated by taking into account the flow rate, culture medium viscosity (0.0078 dyn·s/cm²)(Bacabac, R. G. et al, 2005), and the volume (19.6 mm³), void fraction (0.94), and mean pore radius (8.5 μm, FIG. 6) of the scaffold. Perfusion of culture medium at 0.1 ml/min evoked a shear stress of around 0.7 dyn/cm², while perfusion at 0.2 ml/min yielded a shear stress of around 1.3 dyn/cm². These values are appropriate for the culture of cardiac cells, as it has been shown that compact and contractile cardiac tissue constructs with high expression of cardiac proteins can be obtained with shear stress values ranging from 0.05 to 1.5 dyn/cm² (refs. (Shachar, M. et al, 2012; Cheng et al, 2009)).

Next, the custom-made perfusion chambers including electrical stimulation were installed in the parallelized perfusion bioreactor (FIG. 1d). Parallelization of the culture system allowed the production of multiple cardiac macrotissues under the same physicochemical conditions, and from the same pool of cells. Fluidic restrictors were installed (R_{restrictor}˜10¹³>>R_{manifold}˜10⁷) to ensure that the flow was homogeneously distributed through all branches. Finally, on-line recording of the electrophysiological activity of cardiac tissue constructs was carried out using the two graphite electrodes used for electrical stimulation (one acting as the source and the other one as the reference), and a gold electrode as an internal reference (FIG. 1b). Recorded signals were amplified and noise-filtered to obtain the electrical activity of the constructs.

Structural Signs of Cell and Tissue Maturation in CardioSlice Constructs

To create human cardiac macrotissues, we differentiated cardiomyocytes from hiPSC following a robust and reproducible protocol based on modulation of Wnt/β-catenin signaling with small molecule inhibitors (Lian, X. et al, 2013). hiPSC differentiated in this way formed contracting monolayers comprising high percentages of cardiomyocytes, as defined by co-expression of cardiac troponin I (cTnI) and myosin heavy chain (MHC) proteins (71.6±5.3% cTnI⁺/MHC⁺ cells) after 20 days of differentiation (FIG. 7). In preliminary studies, we determined that fully-differentiated cardiomyocytes (i.e.: after 20 days of differentiation from hiPSC), but not undifferentiated hiPSC or early committed mesoderm progenitors (as defined by the expression of T/Brachyury), could be cultured within the porous scaffolds under continuous perfusion for up to 21 days while maintaining a differentiated phenotype (data not shown). We also verified that addition of fibroblasts to iPSC-derived cardiomyocytes improved the reproducibility of obtaining viable cardiac constructs, in agreement with previous studies (Amano, Y. et al, 2016; Liau, B. et al, 2017; Tiburcy, M. et al, 2017). Based on those preliminary results, for the present studies we used a mixture of hiPSC-derived cardiomyocytes and human dermal primary fibroblasts seeded in a 7:1 ratio. We chose a commercial, clinical-grade porous scaffold made up of collagen and elastin to culture the cells, on account of its biocompatibility and myocardium-like pore size and stiffness (FIG. 6). A total of 5 million cells were seeded into the scaffolds using an on-purpose designed perfusion loop (see details in Material and methods and FIG. 8).

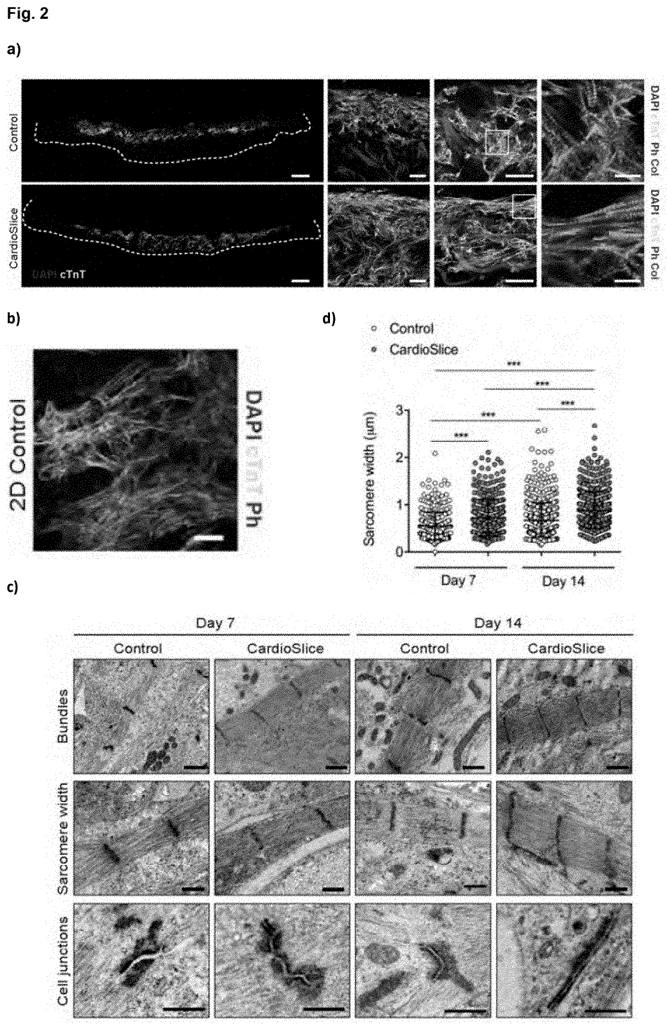

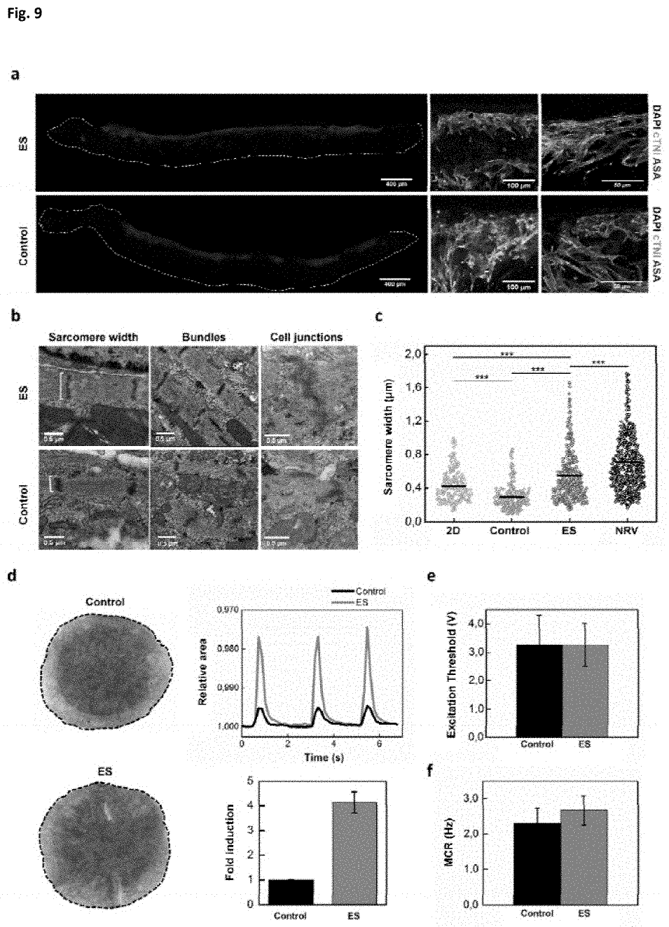

In a first set of experiments, we tested the effects of electrical stimulation on human cardiac macrotissues. For this purpose, constructs were randomly introduced into perfusion chambers and randomly assigned to the control group (only perfusion), or to the CardioSlice group (perfusion and electrical stimulation), and maintained in culture for up to 14 days. Human cardiac macrotissues exhibited cardiomyocytes distributed along the scaffold that strongly expressed cardiac contractile proteins, including cardiac troponin T (cTnT) and α-sarcomeric actin (ASA) (FIG. 2a), with signs of increased cardiomyocyte maturation compared to cells cultured for the same time period under conventional 2D conditions (FIG. 2b). Furthermore, in CardioSlice constructs cardiomyocytes were mostly aligned along the direction of electrical field vector used for stimulation, in contrast with the random distribution found in control constructs (FIG. 2a), a finding also observed in rat-derived cardiac macrotissues (FIG. 9a). At the ultrastructural level, cardiomyocytes in CardioSlice constructs displayed a statistically significant increase in sarcomere width compared with those in control constructs, both after 7 and 14 days in culture (day 7: 0.73±0.39 μm for CardioSlice and 0.55±0.30 μm for control; day 14: 0.88±0.39 μm for CardioSlice and 0.68±0.36 μm for control; p<0.001) (FIG. 2c-d), indicating a higher degree of maturation. Moreover, after 14 days in culture, more developed intercalated discs were detected in CardioSlice versus control constructs (FIG. 2c). This was again in agreement with the improved ultrastructural signs of cardiomyocyte maturation observed in rat-derived cardiac macrotissues, where wider sarcomeres and better developed intercalated discs were present in electrostimulated versus control constructs (FIG. 9b,c).

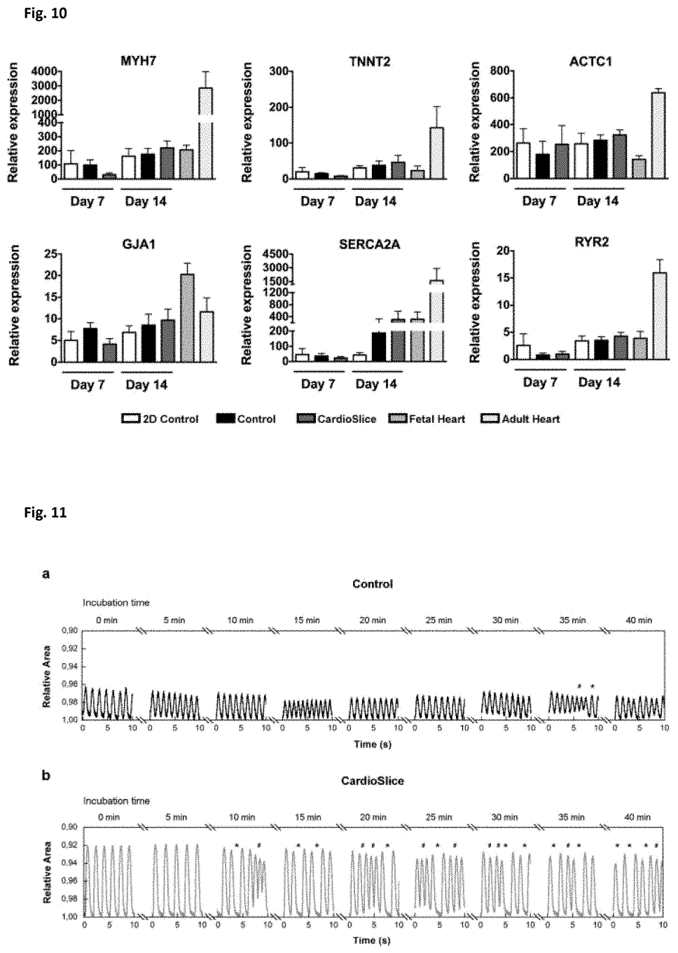

The expression level of key cardiac genes was measured in control and CardioSlice constructs and compared with those of cells cultured for equivalent periods of time under conventional 2D conditions (FIG. 10). As expected, maturity-related cardiac genes (MYH7, ACTC1, TNNT2, MLC2V, GATA4, and GJA1) and cardiac ion channel markers (RYR2, SERCA2A, CACNA1C, SCNA5A, KCNH2, and KCNQ1) were detected in all conditions analyzed. However, no conspicuous differences were noted in cells cultured under 2D or 3D conditions, neither when comparing control and CardioSlice constructs (FIG. 10). Overall, the expression levels of maturity-related cardiac genes and cardiac ion channels were more similar to those of fetal human heart than to those of adult ventricular tissue, consistent with previous analyses of human iPSC-derived cardiomyocytes (reviewed in Yang, X. et al, 2014). These results indicate that any differences observed in the functionality of cardiac macrotissues should be attributed to changes in the maturation of the multicellular structure, rather than to the maturation of its individual cellular constituents.

Biomechanical Signs of Tissue Maturation in CardioSlice Constructs

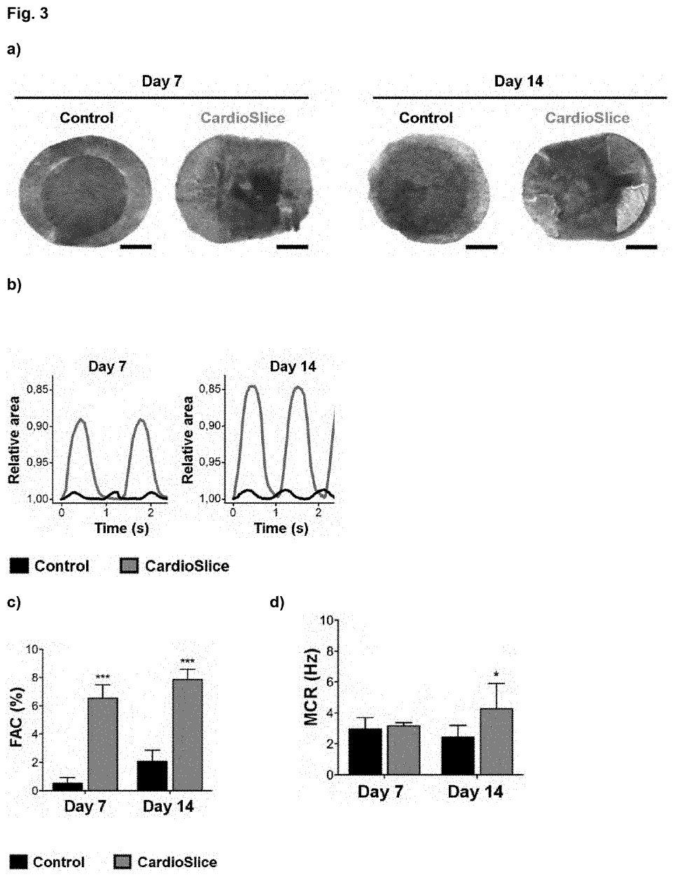

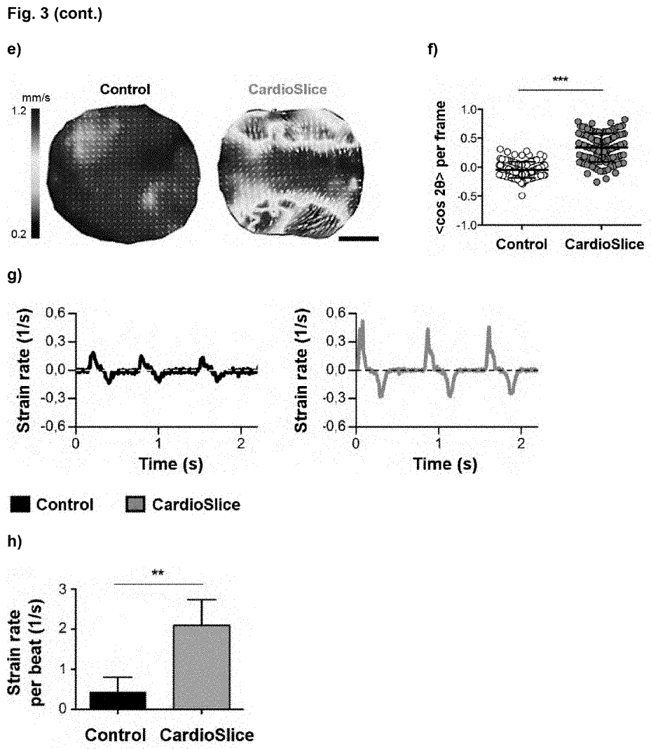

Upon harvesting from the bioreactor chambers, both control and CardioSlice constructs displayed spontaneous beating, which was much more apparent macroscopically in the case of CardioSlice constructs. Electric field stimulation had a direct impact on cell distribution within the CardioSlice constructs, as cells concentrated in the region delimited by the stimulating electrodes (FIG. 3a). The amplitude of contraction, determined by measuring the changes in the fractional area during spontaneous beating, was significantly higher in CardioSlice constructs compared to control ones (day 7: 6.54±0.94% for CardioSlice constructs compared to 0.54±0.09% for control constructs; day 14: 7.85±0.75% for CardioSlice constructs compared to 2.08±0.12% for control constructs; p<0.001), indicating improved maturation of the electromechanical coupling machinery (FIG. 3b, c). Consistently, CardioSlice constructs also exhibited significant higher values for the maximum capture rate (MCR) parameter when compared to control samples. This was evident after 14 days of culture: 4.28±1.63 Hz in CardioSlice constructs versus 2.43±0.75 Hz in control constructs; p<0.001 (FIG. 3d). The effects of continuous electric stimulation were not specific to human cells, since similar improvements in contractile behavior were obtained with rat-derived cardiac constructs (FIG. 9d-f).

The effect of electrical stimulation on the contractility of CardioSlice constructs was further analyzed by Particle Image Velocimetry (NV). The velocity maps generated with this technique demonstrated that the velocity in each contraction was higher in CardioSlice constructs than in controls, corroborating their enhanced contractile performance (FIG. 3e). Directionality of contractions was calculated by determining the alignment of velocity vectors with respect to the direction of the stimulating electric field, and expressed by the order parameter <cos 2θ> (FIG. 3f). CardioSlice constructs contracted in parallel to the direction of the electric field applied during culture (<cos 2θ> close to 1), whereas control constructs displayed random distribution of velocity vectors (<cos 2θ>=0). Regarding the contractile strain generated in each contraction, CardioSlice constructs displayed higher strain rates compared with control constructs (FIG. 3g), with a significantly higher (p<0.01) average strain rate per beat (FIG. 3h). Taken together, these results demonstrate that paced electrostimulation of cardiac macrotissues over the 2 weeks of culture dramatically enhances contractile performance, and suggest improved maturation at the tissue level under these conditions.

CardioSlice Constructs Generate ECG-Like Signals

Human cardiac macrotissues produced a bioelectrical signal that was a sum of the action potentials generated by the individual cardiomyocytes comprising the construct. Such signal could be registered on the surface of constructs using the electrodes embedded within the perfusion chamber (FIG. 4a). In control constructs the recorded electrical signals were of low amplitude and long duration, which is consistent with the incomplete synchronization of contractions visualized across the macrotissue. In contrast, CardioSlice constructs elicited bioelectrical signals composed of narrow and steep waveforms that include the QRS complexes and repolarizing T waves, remarkably similar to those of standard surface ECG for human ventricular myocardium (FIG. 4a). The combined results of 3 independent experiments comparing the bioelectrical signals elicited by control and CardioSlice constructs cultured in parallel for 14 days, showed that the variability in QRS duration and amplitude, as well as in QT interval, was much higher in control than in CardioSlice constructs (FIG. 4b-d). Moreover, ECG-like signals recorded from CardioSlice constructs were of statistically significant higher amplitude, shorter QRS duration, and shorter QT interval than those of control constructs (FIG. 4b-d). These results indicate that cardiomyocytes within CardioSlice constructs are electrically better coupled than within control constructs, attaining the fast conduction velocity and improved action potential propagation characteristic of a functional syncytium.

We next tested whether cardiomyocytes within macrotissue constructs retained the ability to respond to positive and negative inotropic factors. Similar to iPSC-derived cardiomyocytes cultured under standard 2D conditions and to control macrotissue constructs (data not shown), CardioSlice constructs increased or decreased beating rate upon treatment with isoproterenol or carbachol, respectively (FIG. 4e-f). For these experiments, a sterile solution of drug in culture medium was introduced through in-line injection ports, and the effect on the ECG-like signals was recorded over time. The addition of isoproterenol (a standard agonist of β-adrenoceptors) resulted in decreased RR intervals and a statistically significant increment of the beating rate (FIG. 4e). Conversely, treatment with carbachol (standard cholinergic agonist) induced a statistically significant decrease in beating frequency (FIG. 4f).

Electrical Stimulation Improves Structural Organization and Contractile Function of Rat Cardiac Macrotissues

To test the suitability of the bioreactor to generate cardiac macrotissues, a primary culture of neonatal rat cardiomyocytes was used. Tissue constructs were cultured for 7 days under perfusion (Control group) or under perfusion plus electrical stimulation (Electrical stimulation group, ES), and cell morphology and distribution was analyzed by immunofluorescence (FIG. 9a). Both electrostimulated and non-stimulated macrotissues showed positive expression of cardiac markers. However, electrostimulated cardiomyocytes showed a compact and extended distribution along the macrotissue cross-section, whereas the non-stimulated ones formed intermittent groups with high cell densities. Interestingly, cells in electrostimulated tissues displayed abundant and well defined striations aligned in the direction of the electric field, whereas the control ones were scarcer and did not show a preferential direction of alignment (FIG. 9a).

To further characterize cell organization and maturity, cardiac tissues were examined at ultrastructural level by transmission electron microscopy (TEM). Tissue constructs cultured under electrical stimulation showed cardiomyocytes with a well-developed and organized sarcomeric banding, including well-defined Z-bands and intercellular unions composed of desmosomes (FIG. 9b). Moreover, cells in electrostimulated tissues displayed wider sarcomeres in comparison with non-stimulated samples and standard 2D cultures (ES: 0.54±0.32 μm; Control: 0.29±0.16 μm and 2D: 0.42±0.18 μm; P<0.001), suggesting the formation of myofibrillar bundles (FIG. 9c).

Functional activity of rat cardiac macrotissues was also assessed after 7 days in culture by determining the fractional area change (FAC). Cardiac macrotissues exposed to continuous electrical pacing exhibited contraction amplitude values four times higher than non-stimulated ones (FIG. 9d). Moreover, an enhanced contractility with an evident preferential axis of contraction could be observed, as well as a trend towards higher MCR (FIG. 9e,f).

Prediction of Drug-Induced Cardiotoxicity Using CardioSlice Constructs

To investigate the ability of human cardiac macrotissues to predict drug-induced cardiotoxicity, macrotissues at day 14 of culture were incubated with sotalol, a hERG potassium channel blocker and adrenergic antagonist. Upon 10 min treatment, control cardiac macrotissues showed random increases and decreases in beating frequency, and no alteration in the morphology of ECG-like signals (FIG. 5a and FIG. 11a), making it difficult to determine any drug effect. In contrast, CardioSlice constructs displayed a progressive decrease in beating frequency, consistent with sotalol action as adrenergic antagonist (FIG. 5b and FIG. 11b). More importantly, we could detect features typical of arrhythmia, such as ectopic QRS, prolongation of RR intervals and regular blockades (FIG. 5b). These features appeared progressively, were more frequent over time of incubation with sotalol, and coincided with abnormal prolongation of the QT interval, which experienced increases greater than 20% (FIG. 5c). After prolonged incubation times with sotalol, CardioSlice constructs displayed severe alterations in ECG-like signals, with QRS complexes becoming smaller, inverted and even disappearing after 30 min of drug treatment (FIG. 5d). The observed electrophysiological alterations translated, at the mechanical level, into dyssynchronous contractile patterns in sotalol-treated CardioSlice constructs (FIG. 11b). Therefore, sotalol impaired the electrical conduction within CardioSlice constructs and affected their contractile behavior, demonstrating the proarrhythmic potential of the drug. On the whole, these results indicate that CardioSlice constructs are suitable in vitro models for predict drug-induced cardiotoxicity.

Accordingly, the bioreactor system allowed perfusing drugs through the entire tissue, mimicking in vivo microcirculation, which has also been identified as a relevant parameter for testing drug effects (Mathur, A. et al, 2015). These capabilities proved to be determinant when evaluating the drug-induced toxicity effects on our macrotissues. Typically, safety of cardiac drugs is assessed by measuring currents on hERG ion channels ectopically expressed in non-cardiac cell lines (HEK293, CHO). These assays produce false positives and false negatives, as in the cases of verapamil and alfuzosin, respectively (Mathur, A. et al, 2015). Our system allowed to determine the effects of proarrhythmic drugs on the ECG-like signal generated by CardioSlice constructs. Upon treatment with the proarrhythmic drug sotalol, CardioSlice constructs showed a pathological prolongation of the QT interval, alterations in the morphology and polarity of the QRS complex in ECG-like signals and arrhythmias, as it is seen in vivo (Straus, S. M. et al, 2006). Such responses were not identified in control constructs, even though they contained the same numbers of cardiomyocytes and fibroblasts, cultured in parallel under identical conditions (except, of course, for the continuous electrical stimulation applied to CardioSlice constructs). Because proarrhythmic behavior of drugs is oftentimes only found after they have been administered to patients, we propose that the predictive capabilities of CardioSlice constructs reflect their higher degree of close-to-human tissue-like functionality.

In summary, we have developed a method and associated technology that allows the production of thick human cardiac macrotissues with tissue-like functionality. CardioSlice constructs showed improved cellular alignment and ultrastructural organization, as well as contractility and synchronization, and were able to predict drug-induced cardiotoxicity. These data illustrate the enhanced tissue-like functionality achieved by our model. The physiological relevance of CardioSlice constructs, together with their parallel and scalable nature, and the on-line electrophysiological monitoring feature, make our technology to be in the forefront of the production of engineered human cardiac macrotissues.

### REFERENCES

1. Chien, K. R., Domian, I. J. & Parker, K. K. Cardiogenesis and the complex biology of regenerative cardiovascular medicine. Science 322, 1494-1497 (2008).
2. Hunter, P. J., Pullan, A. J. & Smaill, B. H. Modeling total heart function. Annu Rev Biomed Eng 5, 147-177 (2003).
3. Fermini, B. & Fossa, A. A. The impact of drug-induced QT interval prolongation on drug discovery and development. Nat Rev Drug Discov 2, 439-447 (2003).
4. Fleischer, S., Shapira, A., Feiner, R. & Dvir, T. Modular assembly of thick multifunctional cardiac patches. Proc Natl Acad Sci USA 114, 1898-1903 (2017).
5. Zimmermann, W. H. et al. Tissue engineering of a differentiated cardiac muscle construct. Circ Res 90, 223-230 (2002).
6. Shimizu, T. et al. Fabrication of pulsatile cardiac tissue grafts using a novel 3-dimensional cell sheet manipulation technique and temperature-responsive cell culture surfaces. Circ Res 90, e40 (2002).
7. Radisic, M. et al. Functional assembly of engineered myocardium by electrical stimulation of cardiac myocytes cultured on scaffolds. Proc Natl Acad Sci USA 101, 18129-18134 (2004).
8. Parsa, H., Ronaldson, K. & Vunjak-Novakovic, G. Bioengineering methods for myocardial regeneration. Adv Drug Deliv Rev 96, 195-202 (2016).
9. Feric, N. T. & Radisic, M. Maturing human pluripotent stem cell-derived cardiomyocytes in human engineered cardiac tissues. Adv Drug Deliv Rev 96, 110-134 (2016).
10. Schaaf, S. et al. Human engineered heart tissue as a versatile tool in basic research and preclinical toxicology. PLoS One 6, e26397 (2011).
11. Nunes, S. S. et al. Biowire: a platform for maturation of human pluripotent stem cell-derived cardiomyocytes. Nat Methods 10, 781-787 (2013).
12. Thavandiran, N. et al. Design and formulation of functional pluripotent stem cell-derived cardiac microtissues. Proc Natl Acad Sci USA 110, E4698-4707 (2013).
13. Ma, Z. et al. Self-organizing human cardiac microchambers mediated by geometric confinement. Nat Commun 6, 7413 (2015).
14. Tandon, N. et al. Electrical stimulation systems for cardiac tissue engineering. Nat Protoc 4, 155-173 (2009).
15. Godier-Furnemont, A. F. et al. Physiologic force-frequency response in engineered heart muscle by electromechanical stimulation. Biomaterials 60, 82-91 (2015).
16. Hansen, A. et al. Development of a drug screening platform based on engineered heart tissue. Circ Res 107, 35-44 (2010).
17. Mathur, A. et al. Human iPSC-based cardiac microphysiological system for drug screening applications. Sci Rep 5, 8883 (2015).
18. Amano, Y. et al. Development of vascularized iPSC derived 3D-cardiomyocyte tissues by filtration Layer-by-Layer technique and their application for pharmaceutical assays. Acta Biomater 33, 110-121 (2016).
19. Kurokawa, Y. K. & George, S. C. Tissue engineering the cardiac microenvironment: Multicellular microphysiological systems for drug screening. Adv Drug Deliv Rev 96, 225-233 (2016).
20. Lovett, M., Lee, K., Edwards, A. & Kaplan, D. L. Vascularization strategies for tissue engineering. Tissue Eng Part B Rev 15, 353-370 (2009).
21. Carrier, R. L. et al. Perfusion improves tissue architecture of engineered cardiac muscle. Tissue Eng 8, 175-188 (2002).
22. Radisic, M. et al. Medium perfusion enables engineering of compact and contractile cardiac tissue. Am J Physiol Heart Circ Physiol 286, H507-516 (2004).
23. Radisic, M., Marsano, A., Maidhof, R., Wang, Y. & Vunjak-Novakovic, G. Cardiac tissue engineering using perfusion bioreactor systems. Nat Protoc 3, 719-738 (2008).
24. Barash, Y. et al. Electric field stimulation integrated into perfusion bioreactor for cardiac tissue engineering. Tissue Eng Part C Methods 16, 1417-1426 (2010).
25. Bacabac, R. G. et al. Dynamic shear stress in parallel-plate flow chambers. J Biomech 38, 159-167 (2005).
26. Shachar, M., Benishti, N. & Cohen, S. Effects of mechanical stimulation induced by compression and medium perfusion on cardiac tissue engineering. Biotechnol Prog 28, 1551-1559 (2012).
27. Cheng, M., Moretti, M., Engelmayr, G. C. & Freed, L. E. Insulin-like growth factor-I and slow, bi-directional perfusion enhance the formation of tissue-engineered cardiac grafts. Tissue Eng Part A 15, 645-653 (2009).
28. Lian, X. et al. Directed cardiomyocyte differentiation from human pluripotent stem cells by modulating Wnt/beta-catenin signaling under fully defined conditions. Nat Protoc 8, 162-175 (2013).
29. Liau, B., Jackman, C. P., Li, Y. & Bursac, N. Developmental stage-dependent effects of cardiac fibroblasts on function of stem cell-derived engineered cardiac tissues. Sci Rep 7, 42290 (2017).
30. Tiburcy, M. et al. Defined Engineered Human Myocardium With Advanced Maturation for Applications in Heart Failure Modeling and Repair. Circulation 135, 1832-1847 (2017).
31. Yang, X., Pabon, L. & Murry, C. E. Engineering adolescence: maturation of human pluripotent stem cell-derived cardiomyocytes. Circ Res 114, 511-523 (2014).
32. Xiao, Y. et al. Microfabricated perfusable cardiac biowire: a platform that mimics native cardiac bundle. Lab Chip 14, 869-882 (2014).
33. Ruan, J. L. et al. Mechanical Stress Conditioning and Electrical Stimulation Promote Contractility and Force Maturation of Induced Pluripotent Stem Cell-Derived Human Cardiac Tissue. Circulation 134, 1557-1567 (2016).
34. Soong, P. L., Tiburcy, M. & Zimmermann, W. H. Cardiac differentiation of human embryonic stem cells and their assembly into engineered heart muscle. Curr Protoc Cell Biol Chapter 23, Unit 23 28 (2012).
35. Fennema, E., Rivron, N., Rouwkema, J., van Blitterswijk, C. & de Boer, J. Spheroid culture as a tool for creating 3D complex tissues. Trends Biotechnol 31, 108-115 (2013).
36. Kensah, G. et al. Murine and human pluripotent stem cell-derived cardiac bodies form contractile myocardial tissue in vitro. Eur Heart J 34, 1134-1146 (2013).
37. Zhang, D. et al. Tissue-engineered cardiac patch for advanced functional maturation of human ESC-derived cardiomyocytes. Biomaterials 34, 5813-5820 (2013).
38. Turnbull, I. C. et al. Advancing functional engineered cardiac tissues toward a preclinical model of human myocardium. FASEB J 28, 644-654 (2014).
39. Hinson, J. T. et al. HEART DISEASE. Titin mutations in iPS cells define sarcomere insufficiency as a cause of dilated cardiomyopathy. Science 349, 982-986 (2015).
40. Huebsch, N. et al. Miniaturized iPS-Cell-Derived Cardiac Muscles for Physiologically Relevant Drug Response Analyses. Sci Rep 6, 24726 (2016).
41. Maidhof, R. et al. Biomimetic perfusion and electrical stimulation applied in concert improved the assembly of engineered cardiac tissue. J Tissue Eng Regen Med 6, e12-23 (2012).
42. Kensah, G. et al. A novel miniaturized multimodal bioreactor for continuous in situ assessment of bioartificial cardiac tissue during stimulation and maturation. Tissue Eng Part C Methods 17, 463-473 (2011).
43. Ma, Z. et al. Three-dimensional filamentous human diseased cardiac tissue model. Biomaterials 35, 1367-1377 (2014).
44. Eng, G. et al. Autonomous beating rate adaptation in human stem cell-derived cardiomyocytes. Nat Commun 7, 10312 (2016).
45. Tzatzalos, E., Abilez, O. J., Shukla, P. & Wu, J. C. Engineered heart tissues and induced pluripotent stem cells: Macro- and microstructures for disease modeling, drug screening, and translational studies. Adv Drug Deliv Rev 96, 234-244 (2016).
46. Hirt, M. N., Hansen, A. & Eschenhagen, T. Cardiac tissue engineering: state of the art. Circ Res 114, 354-367 (2014).
47. Liang, P. et al. Drug screening using a library of human induced pluripotent stem cell-derived cardiomyocytes reveals disease-specific patterns of cardiotoxicity. Circulation 127, 1677-1691 (2013).
48. Pradhapan, P., Kuusela, J., Viik, J., Aalto-Setala, K. & Hyttinen, J. Cardiomyocyte MEA data analysis (CardioMDA)—a novel field potential data analysis software for pluripotent stem cell derived cardiomyocytes. PLoS One 8, e73637 (2013).
49. Nguemo, F. et al. In vitro model for assessing arrhythmogenic properties of drugs based on high-resolution impedance measurements. Cell Physiol Biochem 29, 819-832 (2012).
50. Yan, P. et al. Palette of fluorinated voltage-sensitive hemicyanine dyes. Proc Natl Acad Sci USA 109, 20443-20448 (2012).
51. Straus, S. M. et al. Prolonged QTc interval and risk of sudden cardiac death in a population of older adults. J Am Coll Cardiol 47, 362-367 (2006).
52. Tandon, N. et al. Optimization of electrical stimulation parameters for cardiac tissue engineering. J Tissue Eng Regen Med 5, e115-125 (2011).
53. Richards-Kortum, R. & Sevick-Muraca, E. Quantitative optical spectroscopy for tissue diagnosis. Annu Rev Phys Chem 47, 555-606 (1996).
54. Jiang, X. et al. Two-photon fluorescence and second-harmonic generation imaging of collagen in human tissue based on multiphoton microscopy. Scanning 33, 53-56 (2011).
55. Thielicke, W. & Stamhuis, E. J. PIVlab—Towards User-friendly, Affordable and Accurate Digital Particle Image Velocimetry in MATLAB. Journal of Open Research Software 2(1):e30 (2014).
56. Thielicke, W. & Stamhuis, E. J. PIVlab—Time-Resolved Digital Particle Image Velocimetry Tool for MATLAB (version: 1.41). Journal of Open Research Software (2014).
57. Thielicke, W. The flapping flight of birds: Analysis and application. Phd thesis, Rijksuniversiteit Groningen. [S.1.]: [S.n.] (2014).
58. Braam, S. R. et al. Repolarization reserve determines drug responses in human pluripotent stem cell derived cardiomyocytes. Stem Cell Res 10, 48-56 (2013).
59. Wu, Z. H. & Huang, N. E. Ensemble empirical mode decomposition: a noise-assisted data analysis method. Adv Adapt Data Anal 1, 1-4 (2009).
60. Halim A S et al., Biologic and synthetic skin substitutes: An overview. Indian Journal of Plastic Surgery 43, 23-28 (2010).
61. Chung et al., Human Embryonic Stem Cell lines generated without embryo destruction. Cell Stem Cell, 2, 113-117 (2008).
62. Takahashi et al. Induction of pluripotent stem cells from adult human fibroblasts by defined factors. Cell 131, 861-872 (2007).
63. Graichen R, et al. Enhanced cardiomyogenesis of human embryonic stem cells by a small molecular inhibitor of p38 MAPK. Differentiation; 76:357-370 (2008).
64. Yang L, et al. Human cardiovascular progenitor cells develop from a KDR+ embryonic-stem-cell-derived population. Nature; 453:524-528 (2008).
65. Kattman S J, et al. Stage-specific optimization of activin/nodal and BMP signaling promotes cardiac differentiation of mouse and human pluripotent stem cell lines. Cell Stem Cell; 8:228-240 (2011).
66. Mohr J C, et al. The microwell control of embryoid body size in order to regulate cardiac differentiation of human embryonic stem cells. Biomaterials; 31:1885-1893. (2010).
67. Azarin S M, et al. Modulation of Wnt/beta-catenin signaling in human embryonic stem cells using a 3-D microwell array. Biomaterials; 33:2041-2049 (2012).
68. Lian X, et al. Robust cardiomyocyte differentiation from human pluripotent stem cells via temporal modulation of canonical Wnt signaling. Proc Natl Acad Sci USA; 109:E1848-1857 (2012).
69. Zhang J, et al. Extracellular Matrix Promotes Highly Efficient Cardiac Differentiation of Human Pluripotent Stem Cells: The Matrix Sandwich Method. Circ Res. 111(9):1125-36 (2012).
70. Ban H et al. Efficient generation of transgene-free human induced pluripotent stem cells (iPSCs) by temperature-sensitive Sendai virus vectors. Proc Natl Acad Sci USA. 23; 108(34):14234-9 (2011).
71. Yu J. et al. Human induced pluripotent stem cells free of vector and transgene sequences. Science 324(5928):797-801 (2009).
72. Warren L. et al. Highly efficient reprogramming to pluripotency and directed differentiation

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for producing a human tridimensional macroscale cardiac construct, wherein said method comprises the following steps:
(i) differentiating human pluripotent stem cells (hPSCs) or cardiac stem cells into contracting cardiomyocytes;
(ii) suspending the contracting cardiomyocytes together with human fibroblasts to obtain a mixed cell suspension;
(iii) seeding the mixed cell suspension into a collagen-based porous scaffold to obtain a seeded scaffold;
(iv) optionally, culturing the seeded scaffold under conditions that allow cell attachment to the collagen-based porous scaffold; and
(v) transferring the seeded scaffold to a bioreactor and culturing the seeded scaffold under perfusion with electrical stimulation for cardiomyocyte maturation,
thereby obtaining a human tridimensional macroscale cardiac construct displaying spontaneous beating,
wherein the human tridimensional macroscale cardiac construct has a thickness greater than 300 μm.

2. The method according to claim 1, wherein said hPSCs in step (i) are induced pluripotent stem cells (iPSCs).

3. The method according to claim 1, wherein said differentiating in step (i) is conducted in a monolayer culture, and the contracting cardiomyocytes obtained in step (i) are disaggregated and suspended in step (ii).

4. The method according to claim 1, wherein said contracting cardiomyocytes obtained in step (i) co express cardiac Troponin T (cTnT) and myosin heavy chain (MHC).

5. The method according to claim 1, wherein in step (ii) said human fibroblasts are dermal skin fibroblasts.

6. The method according to claim 1, wherein in step (ii) said contracting cardiomyocytes and human fibroblasts are at a ratio from 10:1 to 5:1.

7. The method according to claim 1, wherein the collagen-based porous scaffold in step (iii) is a collagen and elastin-based porous scaffold.

8. The method according to claim 1, wherein the collagen-based porous scaffold in step (iii) has macropores with a mean pore size in the range of 50 to 90 μm and micropores with a mean pore size in the range of 5 to 50 μm.

9. The method according to claim 1, wherein the collagen-based porous scaffold in step (iii) is a hydrated scaffold.

10. The method according to claim 1, wherein the collagen-based porous scaffold in step (iii) is between 5 and 50 mm in diameter and between 0.5 and 4 mm in thickness in the hydrated form.

11. The method according to claim 1, wherein the seeding in step (iii) is conducted by perfusion seeding.

12. The method according to claim 1, wherein 5 million or more total cells are seeded in step (iii).

13. The method according to claim 1, wherein in step (iv) the seeded scaffold is cultured in ultralow attachment dishes.

14. The method according to claim 1, wherein in step (iv) the seeded scaffold is cultured for 2-4 hours.

15. The method according to claim 1, wherein during step (v) perfusion of fresh oxygenated culture medium is conducted at a flow rate per chamber of 0.1 or 0.2 ml/min.

16. The method according to claim 1, wherein in step (v) the seeded scaffold is cultured under perfusion for 3 days and under perfusion and electrical stimulation from day 4 onwards.

17. The method according to claim 1, wherein in step (v) the seeded scaffold is subjected to an electric field of about 400 V/m and a current density of about 600 A/m2.

18. The method according to claim 1, wherein the seeded scaffold is cultured in step (v) for at least 7 days.

19. The method according to claim 1, wherein step (v) is conducted in a perfusion bioreactor comprising one or more culture chambers with electrostimulation capabilities.

20. The method according to claim 19, wherein each bioreactor chamber has two electrodes.

21. A human tridimensional macroscale cardiac construct prepared by the method according to claim 1.

22. The human tridimensional macroscale cardiac construct according to claim 21, wherein said human tridimensional macroscale cardiac construct is substantially free from the collagen based-porous scaffold.

23. The human tridimensional macroscale cardiac construct according to claim 21, wherein said human tridimensional macroscale cardiac construct comprises aligned cells with synchronized beating.

24. A method for treatment of a human subject having cardiac damage, comprising administering the human tridimensional macroscale cardiac construct of claim 21 to the human subject.

25. The method according to claim 24, wherein said human subject has ischemic heart disease.

26. A method for screening or evaluating a compound for cardioprotective or cardiotoxic properties, comprising contacting the compound with the human tridimensional macroscale cardiac construct of claim 21 and determining a cardioprotective or cardiotoxic effect.

27. (canceled)
